(19) United States
(12) Patent Application Publication
(10) Pub. No.: US 2022/0348968 A1
(43) Pub. Date: Nov. 3, 2022

(54) PRETREATMENT WITH SULFUR DIOXIDE AND PH ADJUSTMENT

(71) Applicant: IOGEN CORPORATION, Ottawa (CA)

(72) Inventors: Daniel G. MACDONALD, Ottawa (CA); Jeffrey S. TOLAN, Ottawa (CA); Douglas A. MACKENZIE, Ottawa (CA)

(21) Appl. No.: 17/594,970

(22) PCT Filed: Apr. 2, 2020
(86) PCT No.: PCT/CA2020/050436

§ 371 Date: Nov. 4, 2021

Related U.S. Application Data
(60) Provisional application No. 62/844,955, filed on May 8, 2019.

(51) Int. Cl. C12P 7/10; D21C 1/04; C12P 19/02; C12P 19/14; C08H 8/00; C10G 1/00

(52) U.S. Cl. CPC C12P 7/10; D21C 1/04; C12P 19/02; C12P 19/14; C08H 8/00; C10G 1/00; C12P 2201/00

(57) ABSTRACT

A process for producing a fuel from lignocellulosic biomass is disclosed. The process includes obtaining a feedstock comprising lignocellulosic biomass, pretreating a slurry containing the lignocellulosic feedstock, sulfur dioxide, and optionally a bisulfite salt, at temperatures between 110° C. and 160° C. for at least 30 minutes. The pretreatment includes subjecting the slurry to a pH adjustment, which includes adding alkali to the slurry, removing lignosulfonic acid from the slurry, or a combination thereof.

### CROSS-REFERENCE TO RELATED APPLICATIONS

This application claims benefit of U.S. Provisional Application No. 62/844,955, filed May 8, 2019, which is incorporated herein by reference in its entirety.

### TECHNICAL FIELD

The present disclosure relates generally to pretreatment of lignocellulosic feedstock, and more specifically to a process for producing a fuel that includes pretreatment of the lignocellulosic biomass with sulfur dioxide, and optionally bisulfite salt, where the pretreatment includes a pH adjustment.

### BACKGROUND

Lignocellulosic biomass refers to plant biomass that includes cellulose, hemicellulose, and lignin. Lignocellulosic biomass may be used to produce biofuels (e.g., ethanol, butanol, methane) by breaking down cellulose and/or hemicellulose into their corresponding monomers (e.g., sugars), which can then be converted to the biofuel via microorganisms. For example, glucose can be fermented to produce an alcohol such as ethanol or butanol.

While lignocellulosic biomass can be broken down into sugars solely using various chemical processes (e.g., acid hydrolysis), enzymatic hydrolysis is often the preferred approach for generating glucose from cellulose as it is associated with higher yields, higher selectivity, lower energy costs, and milder operating conditions. However, as a result of the complicated structure of the plant cell wall, the enzymatic digestibility of cellulose in native lignocellulosic biomass is often low unless a large excess of enzyme is used (e.g., lignocellulosic biomass may be considered recalcitrant to biodegradation).

In order to reduce biomass recalcitrance (e.g., open up the structure of the lignocellulosic material, make the cellulose more accessible to the enzymes, and/or generally improve enzymatic digestibility of the cellulose) lignocellulosic biomass may be pretreated, a process which can reduce the amount of enzyme and/or enzymatic hydrolysis time required to convert the cellulose to glucose. For example, pretreatment may affect the hemicellulose-lignin sheathing that encases the cellulose.

Pretreatments such as dilute acid or steam explosion may promote hemicellulose dissolution. However, when process conditions for dilute acid or steam explosion are severe, the hemicellulose may degrade to compounds that are potentially inhibitory to enzymatic hydrolysis. In addition, such processes may result in acid-catalyzed condensation of lignin.

Pretreatments such as alkali, organic solvent (organosolv), or aqueous ammonia may promote lignin dissolution. However, such processes may compromise the recovery of the hemicellulose component or may be relatively expensive (e.g., relative to dilute acid processes). For example, with regard to organsolv type pretreatments, the cost of solvent, the additional steps of removing and/or recovering the solvent (e.g., many organic solvents are potentially inhibiting to enzymes), and/or the potential fire and explosion hazards related to the solvent, may increase the cost of such processes.

Pretreatments based on modified sulfite pulping have been proposed (e.g., SPORL). However, in such pretreatments, there may be a tradeoff between lignin dissolution (e.g., which may increase with increasing pH and/or increasing sulfite concentration) and hemicellulose dissolution (e.g., which may increase with decreasing pH). For example, in U.S. Pat. No. 9,243,364, Zhu et al. disclose a two stage process including a first stage, where the lignocellulosic biomass is subjected to a bisulfite cook where the pH>3 to promote delignification and lignin sulfonation, and a second stage, where the pH of the solution is decreased (e.g., to a pH between 1 and 3 by adding H₂SO₄) in order to promote the depolymerization and dissolution of hemicelluloses.

Pretreatment based on sulfur dioxide and/or sulfurous acid, at relatively low temperatures (e.g., between about 110° C. and about 150° C.) and relatively high amounts of sulfur dioxide (e.g., greater than about 20 wt. % based on dry weight of lignocellulosic biomass) have shown potential for promoting both lignin dissolution and hemicellulose dissolution. For example, in PCT/CA2018/000213, residual xylan was found to be as low as about 10 wt. % when the total amount of sulfur dioxide was 28 or 42 wt. %, based on dry weight of lignocellulosic biomass, and the cooking time was at least 180 minutes, while the lignin dissolution reached or exceeded about 50%.

### SUMMARY

The instant disclosure relates to one or more improvements to a pretreatment based on sulfur dioxide (e.g., sulfurous acid), and optionally a bisulfite salt, at temperatures between about 110° C. and about 160° C. and at relatively high sulfur dioxide concentrations (e.g., greater than about 4 wt. % on liquor). More specifically, the instant disclosure recognizes that as the pretreatment progresses the pH may drop as the result of the formation of lignosulfonic acid, and that adjusting the pH during the pretreatment may improve the pretreatment.

According to one aspect of the invention there is provided a process for producing a fuel from lignocellulosic biomass, the process comprising: (a) obtaining a feedstock comprising lignocellulosic biomass; (b) contacting said feedstock with sulfur dioxide and water to provide a slurry having a sulfur dioxide concentration greater than 4 wt. % on liquor and a pH less than 1.5 measured at ambient temperature; (c) pretreating the slurry to provide a pretreated material comprising cellulose, said pretreating comprising: (i) heating the slurry at one or more temperatures between 110° C. and 160° C., said heating conducted for a time sufficient to produce lignosulfonic acid; (ii) adjusting the pH of the slurry containing lignosulfonic acid, said adjusting comprising removing lignosulfonic acid from the slurry, adding alkali to the slurry, or a combination thereof; (iii) heating the pH adjusted slurry at one or more temperatures between 110° C. and 160° C.; and (iv) optionally, repeatedly performing steps (ii) and (iii) in an alternating fashion; (d) hydrolyzing cellulose in the pretreated material to produce glucose, said hydrolyzing comprising adding cellulase to at least the cellulose; (e) fermenting the glucose to produce a fermentation product, said fermenting comprising adding a microorganism to at least the glucose; and (f) recovering the fermentation product, wherein said fuel comprises the fermentation product.

According to one aspect of the invention there is provided a process for producing a fuel from lignocellulosic biomass comprising: (a) obtaining a feedstock comprising lignocellulosic biomass; (b) providing a slurry comprising the feedstock and sulfur dioxide, and optionally including a bisulfite salt, said slurry having a concentration of sulfur dioxide that is greater than 6 wt. % on liquor and a pH less than 1.5 measured at ambient temperature; (c) pretreating the slurry in a batch digester having a circulation loop to provide a pretreated slurry, said pretreating comprising heating the slurry between 110° C. and 160° C. for at least 30 minutes, wherein during said heating the slurry is subjected to a pH adjustment, said pH adjustment comprising adding alkali to the slurry using the circulation loop, removing lignosulfonic acid from the slurry using the circulation loop, or a combination thereof, said pH adjustment conducted in dependence upon a measured pH of the slurry being heated; (d) obtaining a pretreated material produced from (c), said pretreated material having a solid fraction comprising cellulose and a liquid fraction comprising solubilized hemicellulose; (e) hydrolyzing cellulose in the solid fraction to produce glucose, said hydrolyzing comprising adding cellulase to at least the solid fraction; (f) fermenting the glucose to produce a fermentation product, said fermenting comprising adding a microorganism to at least the glucose; and (g) recovering the fermentation product, wherein the fuel comprises the fermentation product.

### BRIEF DESCRIPTION OF THE DRAWINGS

FIG. 1 is a block flow diagram of a fuel production process according to one embodiment;

FIG. 2 is a block flow diagram of a pretreatment process according to one embodiment; and

FIG. 3 is a block flow diagram of a pretreatment process according to one embodiment.

### DETAILED DESCRIPTION

Certain exemplary embodiments of the invention now will be described in more detail, with reference to the drawings, in which like features are identified by like reference numerals. The invention may, however, be embodied in many different forms and should not be construed as limited to the embodiments set forth herein.

The terminology used herein is for the purpose of describing certain embodiments only and is not intended to be limiting of the invention. For example, as used herein, the singular forms “a”, “an,” and “the” may include plural references unless the context clearly dictates otherwise. The terms “comprises”, “comprising”, “including”, and/or “includes”, as used herein, are intended to mean “including but not limited to”. The term “and/or”, as used herein, is intended to refer to either or both of the elements so conjoined. The phrase “at least one” in reference to a list of one or more elements, is intended to refer to at least one element selected from any one or more of the elements in the list of elements, but not necessarily including at least one of each and every element specifically listed within the list of elements. Thus, as a non-limiting example, the phrase “at least one of A and B” may refer to at least one A with no B present, at least one B with no A present, or at least one A and at least one B in combination. In the context of describing the combining of components by the “addition” or “adding” of one component to another, those skilled in the art will understand that the order of addition is not critical (unless stated otherwise).

The instant disclosure describes a process and/or system wherein lignocellulosic biomass is pretreated with sulfur dioxide, and optionally bisulfite salt, prior to enzymatic hydrolysis. More specifically, it describes a process and/or system where the pH is adjusted during the pretreatment.

Pretreatment with sulfur dioxide at relatively low temperatures (e.g., between 110° C. and 150° C.) and relatively high amounts of sulfur dioxide (e.g., greater than 20 wt. % on dry lignocellulosic biomass) has been discussed in PCT/CA2018/000213 and PCT/CA2018/000217. Advantageously, such pretreatment methods may provide sufficient acidity to target hydrolysis of hemicellulose (e.g., which may open up the structure of the lignocellulosic material for a subsequent hydrolysis), while also providing sufficient sulfur to sulfonate lignin (e.g., and thus promote lignin dissolution).

One consequence of working with such high concentrations of sulfur dioxide and/or bisulfite salts is that it may produce a relatively large amount of lignosulfonic acid, which is a strong acid. For example, it has been found that as such pretreatments progress, the pH may drop from an initial pH of 0.95 to a final pH of 0.62 (e.g., when the pretreatment uses 11.1 wt. % SO₂ on liquor, at 140° C., after 180 mins) While the low pH values resulting from the formation of lignosulfonic acid may promote hemicellulose dissolution, and thus may have been previously viewed as advantageous, it is now recognized that the process may be improved if the pH drop is slowed and/or halted (e.g., for at least part of the pretreatment), and/or if the pH is adjusted after the pretreatment reaction has progressed for a time (e.g., to maintain it within a certain range). Without being bound by any theory, the improvements may relate to reduced risk of lignin condensation, higher bisulfite concentrations, and/or more constant reaction conditions.

At very low pH values there is an increased risk of lignin condensation. The relocation and/or modification of the lignin may affect the amount of lignin that can be removed and/or may affect the enzymatic hydrolysis. For example, lignin may act both as a physical barrier, restricting cellulose accessibility, and as a cellulase non-productive binder. Although lignin condensation is generally more of a concern for dilute acid hydrolysis and/or steam pretreatment than for pretreatment with sulfur dioxide and/or bisulfite salt, it may still be a problem for sulfur dioxide pretreatment when the pH reaches very low levels.

Moreover, at very low pH values, bisulfite (HSO₃⁻), which may play a part in the sulfonation reaction, may not be available in sufficient quantities (e.g., may be limited). For example, at ambient temperature, an aqueous solution of sulfur dioxide may contain about 50% HSO₃⁻ (and about 50% SO₂) when the pH is about 1.85, but only about 10% HSO₃⁻ (and about 90% SO₂) when the pH is about 0.9. Accordingly, as the pretreatment progresses and the pH drops, the amount of HSO₃⁻ in solution may also decrease. This may contribute to a slowing in the degree of sulfonation as the pretreatment reaction progresses, and thus may limit the lignin dissolution. Increasing the pH may increase the amount of HSO₃⁻, and thus may promote sulfonation reactions. This may be particularly advantageous during the later stages of the pretreatment.

In general, since there may be some variability in the feedstock, there may be some variability in the production of lignosulfonic acid. For example, if the feedstock is a woody feedstock, there may be some variability in chip size and/or composition (e.g., bark versus heartwood). Accordingly, there may be some variability in how fast and/or how much the pH drops as a result of the formation of lignosulfonic acid. Since pH is one variable that may affect the severity of pretreatment, this may affect the pretreatment product. By adjusting the pH to maintain it within a certain level, the severity within a certain batch and/or between batches may be more consistent. Accordingly, there may be more consistency in the reaction products.

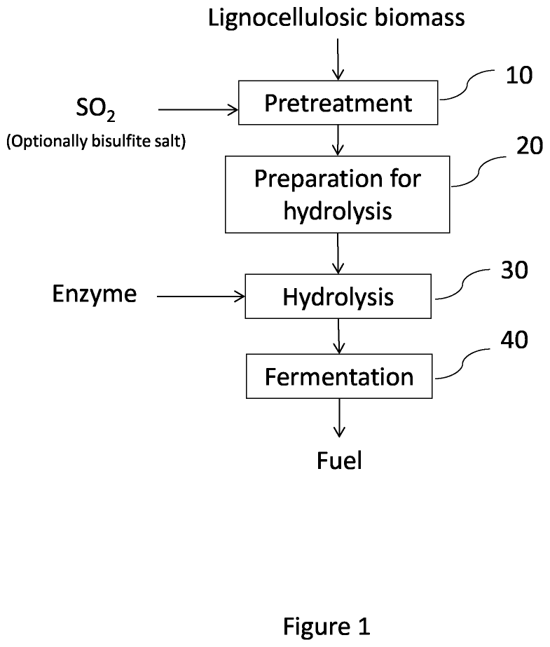

Referring to FIG. 1, there is shown a method in accordance with one embodiment of the invention. Lignocellulosic biomass is subjected to a pretreatment 10, which includes heating the lignocellulosic biomass in the presence of SO₂ and optionally in the presence of a bisulfite salt. The pretreated material is then prepared 20 for hydrolysis (e.g., flashed, filtered, washed, cooled, and/or pH adjusted) and at least the solid fraction thereof is hydrolyzed 30 with added enzyme. The hydrolysis 30 produces sugar (e.g., the cellulose in the pretreated material is converted to glucose). At least the glucose produced during the hydrolysis 30 is fermented 40 (e.g., as part of a separate fermentation step or simultaneous hydrolysis/fermentation), and the fermentation product may be used to produce a fuel.

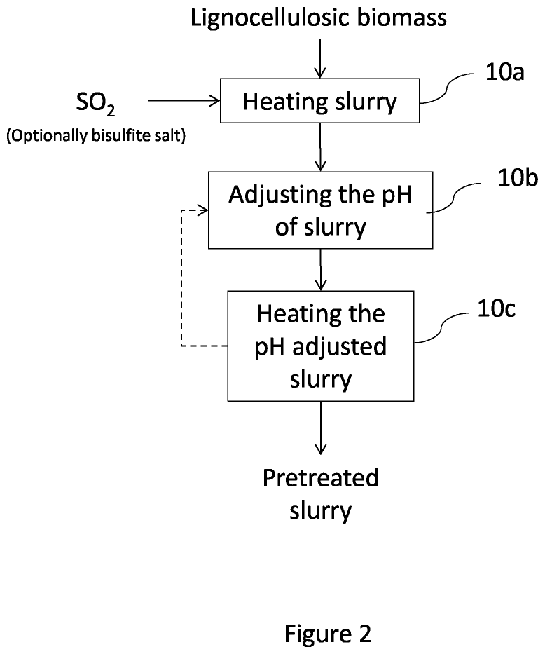

Referring to FIG. 2, the pretreatment 10 may include the steps of heating the slurry 10a, which produces lignosulfonic acid, adjusting the pH of the slurry 10b (e.g., by removing lignosulfonic acid and/or adding alkali), and heating the pH adjusted slurry 10c. Optionally, the step of adjusting the pH is performed iteratively or continuously for the rest of the pretreatment. For example, as illustrated in FIG. 2, the step of adjusting the pH 10b and heating the pH adjusted slurry 10c may be repeated in an alternating fashion.

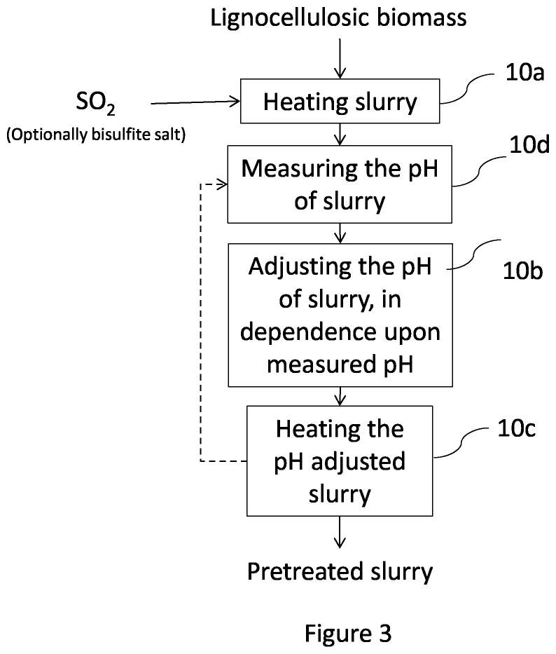

In general, the step of adjusting the pH of the slurry 10b may be initiated at a specific time (e.g., part way through the pretreatment) or in response to a measured parameter. For example, referring to FIG. 3, in one embodiment, the pH is adjusted in dependence upon a pH measured during the pretreatment 10d.

### Feedstock

In one embodiment, the feedstock includes lignocellulosic biomass (e.g., that needs to be pretreated in order to improve enzymatic digestibility). Lignocellulosic biomass may refer to any type of biomass containing cellulose, hemicellulose, and lignin. In one embodiment, the lignocellulosic biomass has a combined content of cellulose, hemicellulose, and lignin that is greater than 25 wt. %, greater than 50 wt. %, or greater than 75 wt. %. In one embodiment, sucrose, fructose, and/or starch are also present, but in lesser amounts than cellulose and hemicellulose.

In one embodiment, the feedstock includes: (i) energy crops; (ii) residues, byproducts, or waste from the processing of plant biomass in a facility or feedstock derived therefrom; (iii) agricultural residues; (iv) forestry biomass; and/or (v) waste material derived from a pulp and paper process.

Energy crops include biomass crops such as grasses, including C4 grasses, such as switch grass, energy cane, sorghum, cord grass, rye grass, miscanthus, reed canary grass, C3 grasses such as Arundo donax, or a combination thereof.

Residues, byproducts, or waste from the processing of plant biomass include residues remaining after obtaining sugar from plant biomass (e.g., sugar cane bagasse, sugar cane tops and leaves, beet pulp, Jerusalem artichoke residue), and residues remaining after grain processing (e.g., corn fiber, corn stover, and bran from grains). Agricultural residues include, but are not limited to soybean stover, corn stover, sorghum stover, rice straw, sugar cane tops and/or leaves, rice hulls, barley straw, wheat straw, canola straw, oat straw, oat hulls, corn fiber, and corn cobs.

Forestry biomass and/or waste material derived from a pulp and paper process includes hardwood, softwood, recycled wood pulp fiber, woodchips, wood pellets, sawdust, trimmings, hog fuel, bark, fines, and/or slash from logging operations.

In one embodiment, the feedstock is a non-woody feedstock. In one embodiment, the feedstock includes an energy or biomass crop. In one embodiment, the feedstock includes an agricultural residue. In one embodiment, the feedstock includes bagasse. In one embodiment, the feedstock includes wheat straw, or another straw. In one embodiment, the feedstock includes stover. In one embodiment, the feedstock is a mixture of fibers that originate from different kinds of plant materials, including mixtures of cellulosic and non-cellulosic feedstock. In one embodiment, the feedstock is a second generation feedstock.

In one embodiment, the feedstock is a woody feedstock. In one embodiment, the feedstock includes hardwood. In one embodiment, the feedstock includes softwood. In one embodiment, the feedstock is obtained from hog fuel, pin chips, and/or other by-products produced by a sawmill. In one embodiment, the feedstock includes wood chips, sawdust, woody shavings, or a combination thereof. In one embodiment, the feedstock includes woodchips that have an average length that is less than 4 cm, less than 3 cm, less than 2 cm, less than 1.5 cm, less than 1.25 cm, less than 1 cm, less than 0.8 cm, less than 0.7 cm, less than 0.6 cm, or less than 0.5 cm. In one embodiment, the feedstock includes woodchips that have an average thickness that is less than 3 cm, less than 2 cm, less than 1.5 cm, less than 1.25 cm, less than 1 cm, less than 0.8 cm, or less than 0.6 cm.

### Feedstock Preparation

In one embodiment, the feedstock is subjected to one or more optional preparatory steps prior to the pretreatment and/or as part of the pretreatment. Some examples of these optional preparatory steps include size reduction, washing, leaching, sand removal, soaking, wetting, slurry formation, dewatering, plug formation, addition of heat, and addition of chemicals (e.g., pretreatment and/or other). In general, these preparatory steps may depend on the type of biomass and/or the selected pretreatment conditions.

In one embodiment, the feedstock is subjected to a size reduction. Some examples of size reduction methods include milling, grinding, agitation, shredding, compression/expansion, and/or other types of mechanical action. Size reduction by mechanical action may be performed by any type of equipment adapted for the purpose, for example, but not limited to, hammer mills, tub-grinders, roll presses, refiners, hydropulpers, and hydrapulpers. In one embodiment, feedstock includes agricultural residue and is subject to a size reduction to yield an average length between about 1/16 inch and about 6 inches. In one embodiment, feedstock includes a woody feedstock and is subject to a size reduction to yield woodchips having an average thickness that is less than 3 cm, less than 2 cm, less than 1.5 cm, less than 1.25 cm, less than 1 cm, less than 0.8 cm, or less than 0.6 cm.

In one embodiment, the feedstock is washed and/or leached with a liquid (e.g., water and/or an aqueous solution). Washing, which may be performed before, during, or after size reduction, may remove sand, grit, fine particles of the feedstock, and/or other foreign particles that otherwise may cause damage to the downstream equipment. Leaching, which may be performed before, during, or after size reduction, may remove soluble components from the feedstock. Leaching may remove salts and/or buffering agents.

In one embodiment, the feedstock is subject to sand removal. For example, in one embodiment, the feedstock is washed to remove sand. Alternatively, or additionally, sand may be removed using other wet or dry sand removal techniques that are known in the art (e.g., including the use of a hydrocyclone or a sieve).

In one embodiment, the feedstock is slurried in liquid (e.g., water), which allows the feedstock to be pumped. In one embodiment, the feedstock is slurried subsequent to size reduction, washing, and/or leaching. The desired weight ratio of water to dry biomass solids in the slurry may be determined by factors such as pumpability, pipe-line requirements, and other practical considerations. In general, slurries having a consistency less than about 10 wt. % may be pumped using a relatively inexpensive slurry pump.

In one embodiment, the feedstock is soaked in water and/or an aqueous solution (e.g., comprising a pretreatment chemical). Soaking the feedstock may allow pretreatment chemical(s) to more uniformly impregnate the biomass, which in turn may provide even cooking in the heating step of pretreatment. For example, soaking the feedstock in a solution comprising a pretreatment chemical (e.g., such as sulfuric acid and/or sulfurous acid) typically provides uniform impregnation of the biomass with the pretreatment chemical. Soaking the feedstock in water, may allow gaseous pretreatment chemicals (e.g., sulfur dioxide) to more uniformly and/or completely impregnate the lignocellulosic biomass during subsequent chemical addition steps. In general, soaking may be carried out at any suitable temperature and/or for any suitable duration.

In one embodiment, the feedstock is wet with a liquid (e.g., water or an aqueous solution) or steam in order to moisten the lignocellulosic biomass and provide a desired consistency. In general, the term consistency refers to the amount of undissolved dry solids or “UDS” in a sample, and is often expressed as a ratio on a weight basis (wt:wt), or as a percent on a weight basis, for example, % (w/w), also denoted herein as wt. %. For example, consistency may be determined by filtering and washing the sample to remove dissolved solids and then drying the sample at a temperature and for a period of time that is sufficient to remove water from the sample, but does not result in thermal degradation of the sample. The dry solids are weighed. The weight of water in the sample is the difference between the weight of the wet sample and the weight of the dry solids.

In one embodiment, the feedstock is at least partially dewatered (e.g., to provide a specific consistency). In one embodiment, the feedstock is at least partially dewatered in order to remove at least some of the liquid introduced during washing, leaching, slurrying, and/or soaking. In one embodiment, dewatering is achieved using a drainer, filtration device, screen, screw press, and/or extruder. In one embodiment, dewatering is achieved using a centrifuge. In one embodiment, the dewatering is achieved prior to and/or as part of plug formation. Some examples of plug formation devices that dewater biomass include a plug screw feeder, a pressurized screw press, a co-axial piston screw feeder, and a modular screw device.

### Pretreatment

In general, the pretreatment includes subjecting the feedstock to a pretreatment with sulfur dioxide. Sulfur dioxide (SO₂) is a gas, which when dissolved in water, may be also referred to as sulfurous acid (H₂SO₃). The term “pretreating” or “pretreatment”, as used herein, refers to one or more steps where the feedstock is treated to improve the enzymatic digestibility thereof (e.g., where the structure of the lignocellulosic biomass is disrupted such that the cellulose in the lignocellulosic biomass is more susceptible and/or accessible to enzymes in a subsequent hydrolysis).

In one embodiment, the pretreatment includes an SO₂ pretreatment. For example, in one embodiment, the pretreatment is an acid pretreatment wherein the lignocellulosic biomass is in contact with SO₂, and wherein to the extent any alkali is added for the pretreatment it is added in an amount that is less than 0.5 wt. % (based on dry weight of incoming lignocellulosic biomass), to the extent any organic solvent is added for the pretreatment it is added in an amount that is less than 5 wt. % (based on dry weight of incoming lignocellulosic biomass), and to the extent any carbonyl compound (or precursor) is added to form α-hydroxysulfonic acid for the pretreatment it is added in an amount less than 0.5 wt. % (based on dry weight of incoming lignocellulosic biomass).

In one embodiment, the pretreatment includes pretreating the lignocellulosic biomass in the presence of SO₂ and bisulfite salt (e.g., HSO₃⁻ salts). As the pretreatment is conducted in the presence of bisulfite salt and SO₂, at low pH values (i.e., below 1.5), it may be referred to as an acid bisulfite pretreatment. The bisulfite salts, which for example may have Na⁺, Ca²⁺, K⁺, Mg²⁺, or NH₄⁺ counter ions, may be added directly (e.g., added as NaHSO₃) and/or may be formed in solution (e.g., by introducing the SO₂ into a solution containing alkali (e.g., a NaOH solution), by adding alkali into a sulfurous acid solution, or by adding sulfite salts to an aqueous SO₂ solution). In one embodiment, the alkali is a hydroxide, an alkali oxide, a sulfite salt, a bisulfite salt, or a lignosulfonate salt.

In one embodiment, the pretreatment includes a pretreatment wherein the lignocellulosic biomass is treated with SO₂ and lignosulfonic acid. The lignosulfonic acid may be generated in situ and/or may be added. Added lignosulfonic acid may be obtained commercially or may be a by-product of the pretreatment process. For example, in one embodiment, the added lignosulfonic acid is introduced into pretreatment when a portion of the pretreated biomass is redirected back to the pretreatment (e.g., as a slip stream). In one embodiment, the lignosulfonic acid is obtained by desalinating a lignosulfonate. For example, in one embodiment, a lignosulfonate produced by the process is contacted with a cation exchange resin to remove cations and recycled back to pretreatment.

In one embodiment, the pretreatment is conducted at a relatively low temperature. In one embodiment, the pretreatment includes heating the lignocellulosic biomass in contact with SO₂ at one or more temperatures between about 110° C. and about 160° C. In one embodiment, the pretreatment includes heating the lignocellulosic biomass with SO₂ at one or more temperatures between about 110° C. and about 150° C. In one embodiment, the pretreatment includes heating the lignocellulosic biomass with SO₂ at one or more temperatures below 150° C. and greater than 120° C., greater than 125° C., greater than 130° C., greater than 135° C., or greater than 140° C. Using pretreatment temperatures between about 110° C. and about 150° C. advantageously avoids the equipment and/or xylose degradation associated with pretreatments at relatively high temperatures (e.g., greater than 160° C.).

In one embodiment, the pretreatment is conducted for at least 30 minutes, at least 45 minutes, or at least an hour. In one embodiment, the pretreatment is conducted for between 30 minutes and 8 hours. In one embodiment, the pretreatment is conducted for between 30 minutes and 7 hours. In one embodiment, the pretreatment is conducted for between 30 minutes and 4 hours. In one embodiment, the pretreatment is conducted for between 1 hour and 3 hours.

In one embodiment, the pretreatment time and/or total amount of SO₂ is selected to convert most of the hemicellulose component to soluble sugars (e.g., xylose, mannose, arabinose, and glucose), but little of the cellulose component to sugars (e.g., which may be hydrolyzed in a subsequent enzymatic hydrolysis). For example, in one embodiment, the degree of pretreatment is selected such that the amount of xylan hydrolyzed to xylose is greater than about 50 wt. %, about 60 wt. %, about 70 wt. %, about 80 wt. %, or about 90 wt. %.

In one embodiment, the pretreatment time and/or total amount of SO₂ provided is selected to provide a pretreatment severity that improves enzyme digestibility of the lignocellulosic biomass. For example, it has been found that when the pretreatment temperature is 130° C., and the total amount of SO₂ is between 20 wt. % and 45 wt. % based on dry weight of lignocellulosic biomass, that enzymatic digestibility of wheat straw is substantially improved when the pretreatment time is greater than 120 minutes, and significantly improved when the pretreatment time is greater than 180 minutes. When the total amount of SO₂ is about 74 wt. % based on dry weight of lignocellulosic biomass, the enzymatic digestibility of wheat straw has been found to be good when the pretreatment time is 180 minutes. In general, providing a pretreatment time that is at least 90 minutes and a total amount of sulfur dioxide that is at least about 25 wt. % based on dry weight of lignocellulosic biomass has been shown to provide good hydrolysis for both wheat straw and bagasse that are washed with water after pretreatment.

The term “total amount of SO₂”, as used herein, refers to the total amount of SO₂ provided for the pretreatment per amount of lignocellulosic biomass on a dry weight basis. In general, the “total amount of SO₂” may be calculated from the grams of SO₂ present initially per gram of dry weight of lignocellulosic biomass present (e.g., as a weight percentage (wt. %)). For example, if 25 g of gaseous SO₂ is added to 100 g of lignocellulosic biomass having total solids (TS) content of 93.25% (e.g., 6.75% moisture content), the total amount of SO₂ is calculated as follows:

$$\begin{matrix}Total amount of SO2 = \frac{g SO2 added}{g biomass added * TS content} = \\ \frac{25 g SO2}{( 100 g biomass \text{*)} * 0.9325} = 27 wt \%\end{matrix}$$

Alternatively, if 52 mL of sulfurous acid prepared to be about 6% (w/w) H₂SO₃ is added to 6.43 g of lignocellulosic biomass having a total solids (TS) content of 93.25% (e.g., 6.75% moisture content), the total amount of SO₂ is calculated as:

$$\begin{matrix}Total amount of SO 2 = \frac{g SO 2 added}{g biomass added * TS content} \\ = \frac{volume H2SO3 ( mL ) added * density of H2SO3 ( \frac{g}{mL} ) * \frac{6 g}{100 g} * \frac{Mw SO2}{Mw H2SO3}}{g biomass added * TS content} \\ = \frac{52 * 1.03 * 6 * 64.066 / ( 100 * 82.07 )}{6.43 * 0.9325} \\ = 42 wt . \%\end{matrix}$$

In some cases, the total amount of SO₂ can be represented by the SO₂ loading. The term “SO₂ loading” is often used for continuous systems, where it refers to the amount of SO₂ fed to the pretreatment system per amount of dry lignocellulosic biomass fed to the pretreatment system (e.g., calculated from the grams of SO₂ provided per gram of dry weight lignocellulosic biomass (e.g., as a weight percentage (wt. %)). However, in some cases, the total amount of SO₂ can be higher than the SO₂ loading (e.g., if some SO₂ is held within the pretreatment system when the pretreated lignocellulosic biomass is discharged). For example, in PCT Application No. PCT/CA2016/051089, filed on Sep. 16, 2016, a pretreatment system having a charge of SO₂ is disclosed. In this case, the total amount of SO₂ provided includes the amount of SO₂ provided in the charge of SO₂.

In some cases, the concentration of SO₂ may include contributions from sulfite salts added to the pretreatment. In general, the SO₂ in the pretreatment may be present as SO₂, H₂SO₃, HSO₃⁻, and/or SO₃²⁻, according to the following reactions:

SO₂+H₂O<=>H₂SO₃ (1)

H₂SO₃+H₂O<=>HSO₃⁻+H₃O⁺ (2)

HSO₃⁻+H₂O<=>SO₃²⁻+H₃O⁺ (3)

However, at the conditions used in the pretreatment (e.g., pH values less than about 1.5), the equilibrium in equation (3) will be shifted to the left and there will be negligible contributions from SO₃²⁻.

In any case, the “concentration of SO₂” or “SO₂ concentration” in pretreatment, which takes into account contributions from SO₂, H₂SO₃, HSO₃⁻, and SO₃²⁻, is expressed on a molar-equivalent-to-SO₂ basis, as weight percent SO₂. The weight percent of SO₂ may be based on the total pretreatment liquid weight (on liquor) or based on the dry lignocellulosic biomass weight (on dry solids). The total pretreatment liquid weight includes the weight of moisture in the feedstock, but not the weight of the dry solids.

In one embodiment, the pretreatment includes contacting the lignocellulosic biomass with SO₂ at one or more temperatures between about 110° C. and about 160° C., where the SO₂ concentration is greater than 15 wt. %, greater than 20 wt. %, greater than 25 wt. %, greater than 30 wt. %, greater than 40 wt. %, greater than 50 wt. %, greater than 60 wt. %, greater than 70 wt. %, greater than 80 wt. %, greater than 90 wt. %, or greater than 100 wt. % (i.e., w/w based on dry weight of lignocellulosic biomass). In one embodiment, the pretreatment includes contacting the lignocellulosic biomass with SO₂ at one or more temperatures between about 110° C. and about 160° C., where the SO₂ concentration is between 20 wt. % and 125 wt. % on dry solids. In one embodiment, the pretreatment includes contacting the lignocellulosic biomass with SO₂ at one or more temperatures between about 110° C. and about 160° C., for more than about 180 minutes, where the total amount of SO₂ is greater than 20 wt. % and less than 125 wt. %, based on dry weight lignocellulosic biomass.

In one embodiment, the pretreatment includes contacting the lignocellulosic biomass with SO₂ at one or more temperatures between about 110° C. and about 160° C., where the SO₂ concentration is greater than 4 wt. %, greater than 6 wt. %, greater than 7 wt. %, greater than 8 wt. %, greater than 9 wt. %, or greater than 10 wt. % (on liquor). In one embodiment, the pretreatment includes contacting the lignocellulosic biomass with SO₂ at one or more temperatures between about 110° C. and about 160° C., where the SO₂ concentration is between 7.8 and 10.4 wt. % (e.g., on liquor). Providing an SO₂ concentration that is greater than 6 wt. % on liquor has been found to be particularly advantageous when the feedstock is a woody feedstock, when significant amounts of alkali have been added, and/or when a pretreatment time under 3 hours is desired. In one embodiment, the pretreatment includes contacting the lignocellulosic biomass with SO₂ at one or more temperatures between about 110° C. and about 160° C., where the H₂SO₃ concentration is greater than 8 wt. % (on liquor), or between 10 and 13 wt. % (on liquor).

In one embodiment, pretreatment includes contacting the lignocellulosic biomass with SO₂ at one or more temperatures between about 110° C. and about 160° C., for a time sufficient to solubilize at least 50 wt. %, at least 55 wt. %, at least 60 wt. %, at least 65 wt. %, at least 70 wt. %, at least 75 wt. %, or at least 80 wt. % of the lignin initially present in the lignocellulosic biomass. In one embodiment, pretreatment includes contacting the lignocellulosic biomass with SO₂ at one or more temperatures between about 110° C. and about 150° C., for a time sufficient to solubilize at least 80 wt. %, at least 85 wt. %, at least 90 wt. %, or at least 95 wt. % of the hemicellulose initially present in the lignocellulosic biomass.

Advantageously, low temperature SO₂ pretreatment has been found to provide good lignin solubilization, good hemicellulose hydrolysis, and good glucose yield without having to add the amount of alkali associated with sulfite pulping based pretreatments and/or without having to add an amount of organic solvent associated with an organosolv process (e.g., to facilitate lignin removal). However, it has also been found that pretreating lignocellulosic biomass with SO₂ at high SO₂ concentrations (e.g., greater than 70 wt. % (on dry solids)) can be advantageous when bisulfate salt is present (e.g., when alkali is added). Bisulfite salts may, for example, be formed by reacting an alkali (base) with aqueous SO₂, or by bubbling SO₂ into a solution containing alkali (base). For example, consider the following acid-base reaction:

H₂SO₃+MOH<=>MHSO₃+H₂O (4)

where M may be referred to as the counter cation. Some examples of alkali suitable for use providing the bisulfite salt include NaOH, NaHCO₃, Na₂CO₃, KOH, KHCO₃, K₂CO₃, CaCO₃, MgO, NH₃, etc.

In one embodiment, an aqueous pretreatment liquor is prepared by adding SO₂ and/or alkali to water or an aqueous solution. In general, the alkali may include any compound(s) that forms the desired bisulfite salt when SO₂ is present (e.g., NaHSO₃, KHSO₃, Ca(HSO₃)₂, Mg(HSO₃)₂, or (NH₄)HSO₃). In one embodiment, the alkali includes NaOH, NaHCO₃, Na₂CO₃, KOH, KHCO₃, K₂CO₃, CaCO₃, CaO, MgO, or NH₃. In one embodiment, the alkali is NaOH, CaO, MgO, or NH₄OH.

The amount of alkali added (e.g., NaOH or CaO) can be expressed as the weight of alkali per dry weight of lignocellulosic solids (on dry solids). For example, if 0.4 g of NaOH is added to 100 g of lignocellulosic biomass having total solids (TS) content of 93.25% (e.g., 6.75% moisture content), the amount of alkali added is calculated as:

$$\begin{matrix}Amount of alkali added = \frac{g alkali added}{g biomass added * TS content} = \\ \frac{0.4 g}{( 100 g biomass \text{*)} * 0.9325} = 0.43 wt \% on dry solids\end{matrix}$$

As the alkali may be provided as a hydroxide, carbonate salt, or other form, for comparative purposes, the “concentration of alkali” or “alkali concentration” may be expressed on a molar-equivalent-to-M basis, where M is the cation on a monovalent basis (Na⁺, K⁺, NH₄⁺, ½Ca²⁺, ½ Mg²⁺), but expressed as weight percent hydroxide (OH).

In one embodiment, the amount of alkali added will be less than about 0.5 wt. % based on dry weight of lignocellulosic biomass. In one embodiment, the amount of alkali added for pretreatment is less than 0.4 wt. % or less than 0.25 wt. % (on dry solids). In one embodiment the amount of alkali added for pretreatment corresponds to a bisulfite loading that is less than 1 wt. % or less than 0.5 wt. % (on dry solids). In one embodiment, the amount of bisulfite salt formed for pretreatment is less than 2 wt. %, or less than 1 wt. % (on dry solids).

In one embodiment, sufficient alkali is added to provide an alkali concentration, near the start of pretreatment, that is at least about 0.05 wt. %, at least about 0.1 wt. %, at least about 0.2 wt. %, at least about 0.3 wt. %, at least about 0.4 wt. %, or at least about 0.5 wt. %, each expressed as weight percent hydroxide on liquor (e.g., OH, on liquor). In one embodiment, sufficient alkali is added to provide an alkali concentration that is between about 0.01 wt. % (OH, on liquor) and about 0.7 wt. % (OH, on liquor). In one embodiment, sufficient alkali is added to provide an alkali concentration that is between about 0.05 wt. % (OH, on liquor) and about 0.5 wt. % (OH, on liquor). In one embodiment, sufficient alkali is added to provide an alkali concentration that is between about 0.1 wt. % (OH, on liquor) and about 0.3 wt. % (OH, on liquor). In one embodiment, sufficient alkali is added to provide an alkali concentration, near the start of pretreatment, between about 0 wt. % and less than about 0.42 wt. % (OH, on liquor).

The alkali concentration on liquor may be converted to the alkali on dry solids by taking the solids consistency into account. In one embodiment, sufficient alkali is added to provide an alkali concentration, near the start of pretreatment, that is at least about 0.10 wt. %, at least about 0.5 wt. %, at least about at least about 1 wt. %, at least about 1.5 wt. %, at least about 2 wt. %, at least about 2.5 wt. %, at least about 3 wt. %, at least about 3.5 wt. %, at least about 4 wt. %, at least about 5 wt. %, or at least about 6 wt. %, each expressed as weight percent hydroxide on dry solids (e.g., OH, on dry solids). In one embodiment, sufficient alkali is added to provide an alkali concentration, near the start of pretreatment, between about 0.50 wt. % and about 3 wt. % (OH, on dry solids).

For reference, if alkali is provided only by adding NaOH, an alkali concentration of about 0.16 wt. % (OH, on liquor) may be roughly equivalent to a NaOH charge of about 0.38 wt. % (on liquor) or a NaHSO₃ charge of about 1 wt. % (on liquor). A NaHSO₃ charge of about 1 wt. % (on liquor) corresponds to a NaHSO₃ charge of about 9 wt. % (on dry solids) when the consistency is about 10 wt. %, about 4 wt. % (on dry solids) when the consistency is about 20 wt. %, or about 1.5 wt. % (on dry solids) when the consistency is about 40 wt. %.

The alkali concentration in the aqueous pretreatment liquor may include contributions from alkali inherent to the feedstock (e.g., K₂CO₃, CaCO₃, and/or Na₂CO₃) and/or alkali added for the pretreatment (e.g., NaOH, CaO, MgO, NH₃, etc.). For example, without adding alkali and without washing, wheat straw may have an inherent alkali concentration that is between about 0.15 wt. % and about 0.63 wt. % (OH, on dry solids), whereas bagasse may provide an inherent alkali concentration as high as about 0.2 wt. % (OH, on dry solids). Woody feedstock tends to have a much lower inherent alkali concentration (e.g., may be negligible).

In one embodiment, alkali is provided via a recycle or backset stream. For example, in one embodiment, compounds derived from the native lignocellulosic feedstock are introduced into pretreatment via a recycle stream (e.g., leach water may be high in potassium bicarbonate). When calculating the amount of alkali added with these compounds for pretreatment (e.g., less than 0.5 wt. % based on dry weight of lignocellulosic biomass), the amount of equivalent OH alkali chemical provided for pretreatment is used.

In one embodiment, alkali is added for the pretreatment in an amount in the range from 0 to 0.5 wt. % based on dry weight of incoming lignocellulosic biomass. In one embodiment, organic solvent is added for the pretreatment in an amount in the range from 0 to 5 wt. % based on dry weight of incoming lignocellulosic biomass. In one embodiment, carbonyl compound (e.g., aldehyde), or precursor, for forming α-hydroxysulfonic acid is added for the pretreatment in an amount in the range from 0 to 0.5 wt. % based on dry weight of incoming lignocellulosic biomass.

The pH (e.g., of the pretreatment liquor and/or the slurry in the pretreatment reactor) may be dependent on the amount of SO₂ (and/or other acids) and/or the amount of alkali present. In one embodiment, the pretreatment liquor is prepared by adding alkali to water or to an aqueous solution of SO₂ such that the ratio of SO₂ to alkali results in excess SO₂ (e.g., such that the pH is below about 1.5).

In one embodiment, sufficient SO₂ is added to provide an initial pH less than 1.5, less than 1.4, less than 1.3, less than 1.25, less than 1.2, less than 1.15, less than 1.1, less than 1.05, or less than 1.0, measured at ambient temperature. The initial pH reflects the pH near the start of pretreatment after the SO₂ has been added to the lignocellulosic biomass (i.e., measured at ambient temperature).

In one embodiment, sufficient SO₂ is added to provide a final pH less than 1.25, less than 1.1, less than 1, less than 0.9, or less than 0.8, measured at ambient temperature. The final pH may be measured after the pretreated material is discharged from the pretreatment reactor (or the last pretreatment reactor if multiple reactors are used in series). In embodiments where the pretreated biomass has a large undissolved solids content and/or is relatively thick, the final pH is measured from a filtrate, pressate, or centrate of the sample (e.g., or other liquid from a solids-liquid separation). In practice, the final pH can be lower than the initial pH. Without being limited by theory, this decrease in pH is attributed to the formation of lignosulfonic acid.

In one embodiment, the pH (e.g., of pretreatment liquor and/or initial) is achieved by selecting an appropriate ratio of SO₂ to alkali. In one embodiment, the ratio of the concentration of SO₂ to concentration of alkali, where the concentration of alkali is expressed as weight percent hydroxide, is greater than 30, greater than 35, greater than 40, greater than 45, or greater than 50.

In one embodiment, the alkali concentration is limited to less than about 0.42 wt. % (OH, on liquor), while the amount of SO₂ provided is sufficient to provide an initial pH less than 1.3. Providing an alkali concentration between 0 and about 0.42 wt. % (OH, on liquor), facilitates and/or improves SO₂ recovery. Providing an alkali concentration between about 0.1 wt. % and about 0.2 wt. % (OH, on liquor), can provide an improved enzymatic hydrolysis.

The concentration of SO₂ (on liquor, or dry solids) may be determined using titration (e.g., with potassium iodate). However, as this may be challenging when relatively high SO₂ concentrations are achieved by introducing SO₂ into a pressurizable reactor, the concentration of SO₂ may be determined using the SO₂ loading. If the reactor has a large headspace (e.g., greater than 75% of the total reactor volume), the concentration of SO₂ can take into account the volume of the reactor headspace and partitioning of SO₂ into the vapour phase.

The concentration of alkali (on liquor, or dry solids), may be determined using the mass of alkali added to pretreatment and/or the mass of inherent alkali. For example, for lignocellulosic biomass that does not contain significant amounts of inherent alkali (e.g., pine), the concentration of alkali may be determined solely using the amount of alkali added to the pretreatment. For lignocellulosic biomass that contains significant amounts of inherent alkali, the alkali concentration may be determined using the amount of alkali added to the pretreatment, in addition to the amount of alkali inherent to the lignocellulosic biomass. The amount of alkali inherent to the lignocellulosic biomass may be determined by preparing a solution of sulfuric acid (H₂SO₄) in water at pH 1.05, 25° C., adding the feedstock to a weight of 5% (dry basis), measuring the resulting pH, and calculating from the acid-base equilibrium of H₂SO₄ the weight of OH as a percentage of the weight of feedstock.

In general, the SO₂, alkali, bisulfite salt, water, and/or feedstock may be added in any order, or simultaneously, to the pretreatment reactor. For example, the aqueous pretreatment liquor may be prepared prior to being introduced to the pretreatment reactor, within the pretreatment reactor, or a combination thereof. In one embodiment, an aqueous pretreatment liquor containing SO₂, alkali, and water is prepared in one or more vessels prior to being introduced into the pretreatment reactor (e.g., which may or may not contain the feedstock).

In one embodiment, an aqueous pretreatment liquor is prepared by adding SO₂ to water, to an aqueous solution containing alkali, to an aqueous bisulfite salt solution, or to an aqueous slurry containing the feedstock. In general, the SO₂ may be added as a gas, as an aqueous solution, or as a liquid (e.g., under pressure). In one embodiment, the aqueous pretreatment liquor is prepared by adding commercially sourced SO₂, by adding SO₂ prepared on site (e.g., by burning sulfur), by adding recycled SO₂ (e.g., recovered from and/or reused within the process), by adding make-up SO₂ (e.g., used to top up the amount of SO₂ present), or any combination thereof. Optionally, the aqueous pretreatment liquor is prepared by adding one or more other acids (e.g., H₂SO₄, HCl, or lignosulfonic acid (LSA)) in addition to the SO₂.

Preparing an aqueous pretreatment liquor containing SO₂ and alkali prior to introducing it into the pretreatment reactor may facilitate providing higher SO₂ concentrations and/or pre-warming of the pretreatment liquor. In general, the concentration of a SO₂ solution may be limited by the solubility of SO₂ in water. For example, if no alkali is added, the SO₂ concentration may be limited to below about 10 wt. % (on liquor) at about 23° C. The SO₂ concentration may be increased by cooling the water or aqueous alkali solution prior to bubbling in SO₂. Alternatively, or additionally, a higher SO₂ concentration may be obtained by introducing the SO₂ under pressure. In one embodiment, SO₂ is introduced into a vessel to provide an SO₂ partial pressure of about 18 psia to about 37 psia, at 25° C. In any case, the pretreatment liquor may or may not be heated prior to entering the pretreatment reactor (e.g., heated under pressure).

In one embodiment, the aqueous pretreatment liquor is prepared using one or more vessels prior to being introduced into the pretreatment reactor (or first reactor if multiple reactors are used). For example, in one embodiment, the aqueous pretreatment liquor is prepared using one or more tanks. In one embodiment, the aqueous pretreatment liquor is prepared using an accumulator, surge tank, and/or buffer tank. Accumulators (or surge tanks), may for example, be used to collect relief gases (e.g., rich in SO₂) for direct reuse. Such relief gases may result if it is necessary to vent the pretreatment reactor.

In one embodiment, the aqueous pretreatment liquor is prepared by feeding SO₂ into water or an aqueous solution containing alkali contained in some vessel (e.g., absorption tower). In one embodiment, SO₂ is bubbled into a cooled alkali solution. In one embodiment, this SO₂/alkali solution is transferred to a pressure accumulator where heat, steam, and/or additional SO₂ (e.g., from a relief valve) are added. In one embodiment, the heated pretreatment liquor from the accumulator is introduced into the pretreatment reactor containing the feedstock. In one embodiment, the feedstock is pre-steamed prior to adding the heated pretreatment liquor. In one embodiment, the feedstock is not pre-steamed prior to adding the heated pretreatment liquor. In one embodiment, the preheated pretreatment liquor and feedstock are heated (e.g., to a temperature between about 110° C. and about 160° C.) in the pretreatment reactor.

In one embodiment, a pre-prepared pretreatment liquor (e.g., containing SO₂, alkali, and water) and the feedstock are introduced into the pretreatment reactor in a liquor to solid ratio (L/kg) of 9:1, 8:1, 7:1, 6:1, 5:1, 4:1, 3:1, 2:1 or 1.5:1. In one embodiment, the pretreatment is conducted on feedstock having a solids consistency between about 5 wt. % and about 51 wt. %. In one embodiment, the pretreatment is conducted on a feedstock having a consistency between about 8 wt. % and about 35 wt. %, between about 12 wt. % and about 25 wt. %, or between about 10 wt. % and 35 wt. %.

Referring again to FIG. 2, once the lignocellulosic feedstock, SO₂, water, and optional alkali (e.g., which forms a bisulfite salt) are added to the pretreatment reactor, the slurry may be heated (e.g., at temperatures between 110° C. and 160° C.). As the slurry is heated, the SO₂ and/or HSO₃⁻ present in solution may sulfonate the lignin. The term “lignin” generally refers to the matrix of phenolic polymers found in the cell walls of many plants. The composition and/or structure of lignin can vary depending on the plant source and/or any methods used to separate it from the cell walls. The term “lignin”, as used herein, refers to the intact lignin structure found in the cell walls of plants and/or fragments or compounds derived therefrom resulting from disruption of the lignin structure. In particular, the term “lignin”, as used herein, includes soluble lignin or derivatives of the lignin, condensed lignin, and insoluble unreacted lignin. The term “sulfonated lignin” refers to lignin into which sulfonic acid groups and/or sulfonate groups have been introduced. For example, sulfonated lignin can be produced by reacting lignin with sulfur dioxide, a bisulfite, and/or or a sulfite at elevated temperatures. The term “lignosulfonate” or “lignosulfonates” refers to water soluble sulfonated lignin (i.e., soluble in water at neutral and/or acid conditions). For example, water solubility may be imparted when sulfonic acid and/or sulfonate groups are incorporated within lignin in an amount effective to solubilize the lignin. The terms “lignosulfonate” or “lignosulfonates”, as used herein, encompasses both lignosulfonic acid and its neutral salts.

After an initial heating period (e.g., 10 to 30 minutes depending on the temperature and SO₂ concentration) the pH of the slurry may begin to drop. Without being bound by theory, this pH drop is believed to be a result of the formation of lignosulfonic acid. The term “lignosulfonic acid” refers to lignosulfonate wherein a significant number of the sulfonate groups on the lignin are protonated (—SO₃H) or fully dissociated (—SO₃⁻), without being bound to a salt-forming cation (e.g., such that a solution or slurry thereof has a pH less than 7). The terms “concentration of lignosulfonic acid” or “concentration of LSA” are interchangeable and refer to the concentration of sulfonate groups on the lignin that are in acid form (i.e., protonated or fully dissociated, and not bound with a salt-forming cation and/or metal). The concentration of lignosulfonic acid may be expressed in moles per liter of solution or slurry.

In accordance with one embodiment of the instant invention, the pH of the slurry is adjusted during the pretreatment. In general, the pH may be adjusted as the slurry is heated or in between heating stages. In one embodiment, the pH is adjusted by removing some of the lignosulfonic acid. For example, in one embodiment, the pH is adjusted by filtering, pressing, and/or washing the slurry to remove some of the lignosulfonic acid. In one embodiment, the removed liquid is replaced with a quantity of water and fresh SO₂ before the slurry is heated again. In one embodiment, the pH is adjusted by adding alkali. In general, any suitable alkali may be added. For example, in one embodiment, the alkali added is NaOH, KOH, K₂CO₃, CaCO₃, CaO, MgO, NH₃, MgO, and/or NH₄OH. In general, the alkali may be added in gaseous, liquid, or solid form. In one embodiment, the alkali is added as an aqueous solution. In one embodiment, the pH of the slurry is adjusted to keep the pH substantially constant (e.g., to counter a pH change as it occurs), to keep the pH above a predetermined limit, or to keep the pH within a predetermined range.

In one embodiment, sufficient lignosulfonic acid is removed and/or sufficient alkali is added to keep the pH of the slurry being pretreated above a predetermined value. For example, in one embodiment, sufficient lignosulfonic acid is removed and/or sufficient alkali is added to keep the pH above 0.6, 0.7, 0.8, 0.9, or 1. Without being bound by theory, keeping the pH above 0.9 may prevent lignin condensation that occurs at longer pretreatment times, and thus may improve hydrolysis.

In one embodiment, sufficient lignosulfonic acid is removed and/or sufficient alkali is added to keep the pH of the slurry being pretreated within a predetermined range throughout the pretreatment. For example, in one embodiment, sufficient lignosulfonic acid is removed and/or sufficient alkali is added to keep the pH between about 0.6 and 1.5, between about 0.7 and 1.4, between about 0.8 and 1.3, between about 0.9 and 1.2, or between about 0.95 and 1.1. Without being bound by theory, keeping the pH above 0.9 and below 1.3 may provide sufficient acidity to promote hemicellulose dissolution without significantly sacrificing lignin dissolution. Keeping the pH at or about 1 (e.g., plus or minus 0.5) may be particularly advantageous as good hydrolysis results have been obtained for pretreatments wherein the initial pH is about 1 (e.g., +/−0.5).

In one embodiment, sufficient lignosulfonic acid is removed and/or sufficient alkali is added to keep the pH of the slurry being pretreated above a predetermined value or within a predetermined range, for part of the pretreatment. For example, in one embodiment, the pH is permitted to drop to some value (e.g., 0.85-0.9) and at later stage of the pretreatment, the pH is increased to between 1.0 and 1.5. Advantageously, this may provide the acidic conditions that promote hemicellulose dissolution and may increase the amount of HSO₃⁻ when sulfonation begins to slow.

In another embodiment, the pH is adjusted earlier in the pretreatment, but allowed to drop to some value (e.g., 0.6-0.8) later in the pretreatment. Advantageously, this may slow the pH drop, while providing the low pH values associated with good hemicellulose dissolution once most of the lignin has been solubilized. In one embodiment, the pH is adjusted to keep the pH above a predetermined value until the amount of lignin solubilized reaches a maximum (e.g., substantially levels off), at which time the pH is permitted to drop.

In one embodiment, sufficient lignosulfonic acid is removed and/or sufficient alkali is added to keep the pH of the slurry being pretreated within a certain range of the initial pH. For example, in one embodiment, sufficient lignosulfonic acid is removed and/or sufficient alkali is added to ensure that the pH does not fall to more than 0.1, 0.15, 0.2, 0.25, or 0.3 below the initial pH. For example, in one embodiment, if the initial pH is 1.1, alkali may be added when the pH of the slurry is 0.8, 0.85, 0.9, 0.95, or 1, measured at ambient temperature. In this embodiment, the pH may be adjusted in dependence upon a measured pH of the slurry, as illustrated, for example, in FIG. 3.

In general, the step of adjusting the pH may be performed incrementally or continuously. For example, consider the case where the pH adjustment includes adding alkali. In one embodiment, alkali is added continuously. In this embodiment, the concentration of alkali may be varied to tailor the pH. In one embodiment, alkali is added as one or more discrete additions. For example, in one embodiment, alkali is added at a time and in an amount selected such that a single alkali addition step provides the desired pH adjustment for the entire pretreatment (e.g., following the solid lines in FIGS. 2 and 3). In one embodiment, alkali is added as a plurality of discrete additions (e.g., incrementally). In this embodiment, the amount of alkali may or may not be constant for each addition. Furthermore, the timing between discrete additions may or may not be uniform.

In one embodiment, alkali is added incrementally, and the process includes an alternating cycle of adjusting the pH of the slurry 10b and heating the pH adjusted slurry 10c. For example, referring to FIG. 2, after the slurry has been heated 10a and the pH has dropped, the slurry may be subjected to a pH adjustment 10b. After further heating 10c and an additional drop in pH, the process may follow the dashed lines such that the steps of adjusting the pH of the slurry 10b and heating the pH adjusted slurry 10c are repeated in an alternating pattern. In general, there may be any number of cycles in this alternating pattern. In FIGS. 2 and 3, the process steps of measuring the pH 10d, adjusting the pH 10b, and heating 10c, are shown as separate steps. However, one the art will understand that this does not imply that the heating of the slurry is interrupted between the pH adjustment steps (e.g., alkali may be added as the pH is measured and/or as the slurry is heated).

In general, the step of adjusting the pH of the slurry 10b may be initiated at a specific time (e.g., ¼, ⅓, ½, or ¾ way through the pretreatment) or in response to a measured parameter. For example, referring again to FIG. 3, the step of adjusting the pH of the slurry 10b may be performed in dependence upon the step of measuring the pH of the slurry 10d (e.g., during the pretreatment). In general, the pH of the slurry may be dependent on temperature and/or pressure. Accordingly, the measured pH may correspond to the pH at the pretreatment temperature and pressure or may correspond to the pH of the slurry if it were cooled to ambient temperature at atmospheric pressure. For example, a pH at ambient temperature may be measured by extracting a sample from the pretreatment reactor, cooling it to ambient temperature, and taking the pH measurement. Alternatively, the pH at ambient temperature may be determined using the pH measured at the pretreatment temperature and pressure, which is then corrected to ambient temperature (e.g., using calibration data).

In one embodiment, the step of adjusting the pH may be initiated when the measured pH is at or below a predetermined level. In this embodiment, the measured pH may be the temperature corrected pH or may be the pH measured at the pretreatment temperature and pressure. In one embodiment, the step of adjusting the pH may be initiated when the measured pH drops by a certain amount in a certain amount of time. For example, in one embodiment, the step of adjusting the pH may be initiated when the rate of change in pH exceeds a certain value. Performing the step of adjusting the pH in dependence upon a measured pH advantageously may ensure that the pH does not go below a predetermined limit and/or stays within a predetermined range. Moreover, it may facilitate monitoring a rate of change of the pH and/or slowing the pH drop.

As described above, the pH may be adjusted to counter or off-set the pH drop accompanying the progression of the pretreatment. However, since pH is one parameter that determines the severity of a pretreatment, increasing pH could potentially lengthen the required pretreatment time. Without being bound by theory, it is now believed that adjusting the pH to off-set the pH drop accompanying the progression of the pretreatment can decrease the required pretreatment time because it may keep the lignin susceptible to sulfonation. In contrast, if the pH is permitted to drop to the extent that lignin condenses, then the condensed lignin may not be as susceptible to sulfonation. In any case, adjusting the pH to off-set the pH drop accompanying the progression of the pretreatment, allows a higher concentration of SO₂ to be used in the initial stages of the pretreatment (e.g., which may be advantageous in terms of rapid sulfonation) without subjecting the system to the low final pH values that would normally accompany pretreatment with such high SO₂ concentrations.

In one embodiment, the pretreatment is carried out in batch mode, semi-batch mode, or continuous mode, in one or more pretreatment reactors. For example, the pretreatment may be conducted in one or more vertical reactors, horizontal reactors, inclined reactors, or any combination thereof.

In one embodiment, the pretreatment is carried out in batch mode in a steam autoclave. In one embodiment, the pretreatment is conducted in a plug flow reactor. In one embodiment, the pretreatment is conducted in a continuous mode horizontal screw fed reactor. In one embodiment, the pretreatment is conducted in a counter-current flow reactor. In one embodiment, the pretreatment is conducted in reactor provided with a charge of SO₂ (e.g., as described in PCT Application No. PCT/CA2016/051089). In one embodiment, the pretreatment is conducted in a digester (e.g., batch or continuous). Such digester may be of any suitable conventional construction (e.g., used in wood pulping).

In one embodiment, the pretreatment is conducted in a pretreatment system, which may include a plurality of components/devices in addition to a pretreatment reactor. Some examples of these devices/components include a biomass conveyer, washing system, dewatering system, a plug formation device, a heating chamber, a high shear heating chamber, a pre-steaming chamber, an SO₂ impregnation chamber, vapour reservoir chamber, an additional pretreatment reactor, connecting conduits, valves, pumps, etc.

In one embodiment, the pretreatment is conducted in a pretreatment system and/or reactor that is pressurizable. For example, in one embodiment, the pretreatment reactor and/or pretreatment system includes a plurality of valves and/or other pressure increasing, pressure decreasing, or pressure maintaining components for providing and/or maintaining the pretreatment reactor at a specific pressure. Conventional digesters used in wood pulping are generally pressurizable.

In one embodiment, the pretreatment includes adding steam to provide a total pressure between about 190 psia and about 630 psia, between about 200 psia and about 600 psia, between about 250 psia and about 550 psia, or between about 300 psia and about 500 psia. For example, in one embodiment, where the total pressure is about 190 psia, the partial pressure of SO₂ may be about 21 psia, whereas the steam partial pressure may be about 169 psia.

In one embodiment, the pretreatment is conducted in a pretreatment system and/or reactor that includes a heater, or some other heating means, for heating the feedstock. Such heating may be direct or indirect (e.g., direct steam heating or indirect steam heating). In one embodiment, the pretreatment reactor and/or the pretreatment system includes direct steam injection inlets (e.g., from a low pressure boiler). For example, in one embodiment, the pretreatment reactor is a digester that provides direct steam injection at the bottom of the digester, with heat transfer throughout the rest of the digester occurring by convection. In one embodiment, the pretreatment reactor is heated by indirect steam heating via the use of one or more heat-exchangers and forced liquor circulation (e.g., using liquid circulation loops). For example, in one embodiment, the aqueous pretreatment liquor is removed from the digester through a screen, and returned to the digester via a pipe, after the circulating liquid is heated with a heat exchanger coupled to the pipe.

In one embodiment, the pretreatment is carried out in a batch digester. Batch digesters, which are chemical reactors, are commonly used for delignifying wood chips for kraft pulp production. For example, such batch digesters may be cylindrical, 2.5-5 m in diameter, 8.5-19 m in height, have a height to diameter ratio of about 5:1, and/or a volume between 25-400 m³.

In one embodiment, the pretreatment is carried out in a batch digester with liquid circulation. For example, forced circulation digesters may include an outlet disposed just below the digester midpoint (e.g., equipped with extraction screens) that draws out liquid during the digestion and forces it back into the digester at the top and/or bottom, after heating the extracted liquor by passing it through an indirect steam heater. Advantageously, this type of external loop may be used for monitoring the pH and/or adjusting the pH.

In one embodiment, the pretreatment is carried out in a batch digester with liquid circulation, where the batch digester includes at least one circulation loop (e.g., which may be used to monitor the pH and/or provide heat). In one embodiment, the circulation loop is coupled to a heat exchanger. In one embodiment, the circulation loop includes a sampling port and/or a pH system (e.g., including a pH probe). In one embodiment, the pH system includes an automated monitoring system and/or a controller for actuating pH adjustment. In one embodiment, the pH is monitored using the sampling port. In one embodiment, the circulation loop includes a port for injecting alkali and/or acid during the pretreatment. In one embodiment, the liquid circulation loop is equipped with a pressurized injector for injecting the alkali and/or acid. In one embodiment, the circulation loop is coupled to a pump that a circulation rate sufficient to turn over the volume of digester every 1 to 5 hours.

In one embodiment, the pH is adjusted during the pretreatment by injecting alkali (e.g., as an aqueous solution) into the circulation loop. In one embodiment, the amount of alkali (e.g., volume and/or concentration) is adjusted in dependence upon a measured pH and/or rate of change in pH.

In one embodiment, the pH is adjusted by removing lignosulfonic acid via the circulation loop. For example, in one embodiment, cooking liquor is drawn through the extraction screens into the circulation loop and withdrawn from the system via an outlet on the circulation loop. In this embodiment, water, an aqueous solution of SO₂, or an aqueous solution of SO₂ and bisulfite salt, may be injected (e.g., after being heated) into the circulator loop to replace at least some of the withdrawn cooking liquor that contains lignosulfonic acid. In one embodiment, the withdrawn liquor is reinjected back into the digester at a later stage in the pretreatment (e.g., once most of the lignin has been solubilized) in order to promote hemicellulose dissolution of that batch. In one embodiment, the withdrawn liquor is used for pretreating another batch. In either embodiment, the withdrawn cooking liquor may be processed (e.g., subjected to ion exchange in order to decrease the pH and/or filtered to remove sugars) prior to injection/reuse. Advantageously, in addition to tempering pH drops associated with the progression of the pretreatment, withdrawing the cooking liquor containing lignosulfonic acid may also remove solubilized hemicellulose, and thus may minimize and/or reduce the production of degradation products from hemicellulose sugars. Further advantageously, this pH adjustment is achieved using reaction chemicals that were added initially or generated during the process (e.g., does not introduce additional chemicals).

In one embodiment, the pretreatment is carried out in a batch digester with liquid circulation, and the pH in the digester is adjusted by injecting alkali and acid into the circulation loop. For example, in one embodiment, the process includes adding alkali to off-set the pH drop accompanying the progression of the pretreatment (e.g., until most of the lignin than can be readily solubilized is solubilized), at which point lignosulfonic acid generated from another batch and/or additional SO₂ is injected to reduce the pH and solubilize hemicellulose.

Conducting the pretreatment in a batch digester with liquid circulation is particularly advantageous for pretreating woody feedstocks, and more specifically, for softwoods, which have a relatively high resin content. For example, at low pH values (e.g., below 1.5), the presence of resinous extractives (e.g., phenolic compounds) can favour the condensation of lignin over sulfonation reactions, which may prevent efficient delignification. In particular, the heartwood of pine can contain relatively high amounts of phenolic compounds such as pinosylvin, which may condense with lignin moieties. By adjusting the pH of the cooking liquor, the lignin condensation may be reduced.

In one embodiment, the pretreated material is discharged from the pretreatment reactor under pressure (e.g., blow down). In one embodiment, the discharged pretreated material is collected in a receiving vessel (e.g., a flash tank or blow tank, which may or may not be at atmospheric pressure). In one embodiment, the discharged pretreated material is collected in a diffusion washer. In one embodiment, the discharged pretreated material is fed for downstream processing.

### Preparing the Pretreated Material for Enzymatic Hydrolysis

In general, the pretreated material may be subject to one or more steps to prepare it for hydrolysis. For example, in one embodiment the pretreated material is subject to a pressure reduction (e.g., flashing), a liquid/solid separation (e.g., filtering), a washing step, a cooling step, mechanical refining, and/or a pH adjustment step.

In one embodiment, the pretreated biomass is subject to a pressure reduction. For example, in one embodiment, the pressure is reduced using one or more flash tanks in fluid connection with the pretreatment reactor. Flashing may reduce the temperature of the pretreated biomass to 100° C. if an atmospheric flash tank, or lower if a vacuum flash tank.

In one embodiment, the pretreated biomass is subject to a liquid/solid separation, which provides a solid fraction and a liquid fraction. The solid fraction may contain undissolved solids such as unconverted cellulose and/or insoluble lignin. The liquid fraction, which may also be referred to as the xylose-rich fraction, may contain soluble compounds such as sugars (e.g., mannose, xylose, glucose, and arabinose), organic acids (e.g., acetic acid and glucuronic acid), soluble lignin (e.g., including soluble products of reactions between sulfur dioxide/sulfurous acid and lignin, such as lignosulfonic acids), soluble sugar degradation products (e.g., furfural, which may be derived from C5 sugars, and hydroxymethylfurfural (HMF), which may be derived from C6 sugars) and/or one or more salts (e.g., sulfite salts). Exemplary solid/liquid separation methods include, but are not limited to, filtration, membrane filtration, tangential flow filtration (TFF), centrifugation, sedimentation, and flotation. For example, in one embodiment, the pretreated material fed to one or more centrifuges that provide a solid stream and a liquid stream. In one embodiment, the solid/liquid separation uses vacuum or pressure to facilitate the separation. For example, in one embodiment, the pretreated material fed to a filter press or belt press. In one embodiment, the solid/liquid separation is conducted in batch, continuous, or dis-continuous mode.

In one embodiment, the pretreated biomass is subject to one or more washing steps. For example, in one embodiment, the solid fraction from a solid/liquid separation is washed to remove soluble components, including potential inhibitors and/or inactivators. Washing may also remove lignin (e.g., sulfonated lignin). In one embodiment, the pretreated biomass is washed as part of the liquid/solid separation (e.g., as part of decanter/wash cycle). The pretreated biomass may be washed as part of the liquid/solid separation at high or low pressure, which may or may not reduce the temperature of the pretreated biomass. In one embodiment, the wash water is reused or recycled. In one embodiment, the wash water and the liquid fraction are fed to fermentation. In one embodiment, lignin and/or lignosulfonic acid is extracted from the wash water. In one embodiment, the wash water is combined with the liquid fraction and sent for further processing.

In one embodiment, the pretreated biomass is subjected to one or more cooling steps. For example, in one embodiment, the pretreated biomass is cooled to within a temperature range compatible with enzyme(s) added for the enzymatic hydrolysis. For example, conventional cellulases often have an optimum temperature range between about 40° C. and about 60° C., and more commonly between about 50° C. and 55° C., whereas thermostable and/thermophilic enzymes may have optimum temperatures that are much higher (e.g., as high as, or greater than 80° C.). In one embodiment, the pretreated biomass is cooled to within a temperature range compatible with enzyme(s) and yeast used in a simultaneous saccharification and fermentation (SSF).

In one embodiment, cooling is provided primarily from flashing. In one embodiment, cooling is provided primarily using a heat exchanger. In one embodiment, cooling is provided primarily by washing the solids. In one embodiment, cooling is provided by any combination of flashing, heat exchange, washing, and other cooling techniques. In one embodiment, cooling is provided by injecting a fluid into the pretreated biomass. For example, in one embodiment, cooling is achieved when alkali and/or water is added to the pretreated biomass into order to provide the pH and/or consistency desired for enzymatic hydrolysis.

Advantageously, since the pretreatment is conducted at relatively low temperatures (e.g., between 110° C. and 160° C.), the one or more cooling steps may not have to produce a significant temperature drop.

In one embodiment, the pretreated material is subjected to one or more mechanical refining steps. For example, in one embodiment, the pretreated material (e.g., solid fraction or whole slurry) is subject to a mechanical size reduction using disk refining. Disk refining, may for example, be advantageous when the feedstock includes large woodchips. In one embodiment, disk refining is conducted on the solid fraction after the solid/liquid separation and/or washing.

In one embodiment, the pretreated biomass is subjected to one or more pH adjustment steps. In one embodiment, the pH of the pretreated biomass is adjusted to within a range near the pH optimum of the enzyme(s) used in hydrolysis. For example, cellulases typically have an optimum pH range between about 4 and about 7, more commonly between about 4.5 and about 5.5, and often about 5. In one embodiment, the pH is adjusted to between about 4 and about 8. In one embodiment, the pH is adjusted to between about 4.5 and about 6. In one embodiment, the pH is adjusted so as to substantially neutralize the pretreated biomass.

In one embodiment, the pH of the pretreated biomass is increased as a result of the washing step. In one embodiment, the pH of the pretreated biomass is increased by adding alkali (e.g., calcium hydroxide, potassium hydroxide, sodium hydroxide, ammonia gas, etc.). For example, in one embodiment, sufficient alkali is added to increase the pH of the pretreated biomass to a pH near the optimum pH range of the enzyme(s) used in hydrolysis. In one embodiment, the pH adjustment step includes adding sufficient alkali to overshoot the optimum pH of the enzyme (e.g., overliming), and then adding acid to reduce the pH to near the optimum pH range of the enzyme(s).

In general, the pH adjustment step may be conducted on the solid fraction, the liquid fraction, and/or a combination thereof, and may be conducted before, after, and/or as part of the one or more cooling steps. For example, in embodiments wherein the pretreated biomass is separated into a solid fraction and a liquid fraction, where only the solid fraction is fed to enzymatic hydrolysis, the pH of the liquid fraction may require adjustment prior to being fed to fermentation (e.g., separate from, or with, the hydrolyzate from the solid fraction). For example, in one embodiment, the pH of the liquid fraction is adjusted to the pH optimum of the microorganism used in a subsequent fermentation step. For example, Saccharomyces cerevisiae may have optimum pH values between about 4 and about 5.5.

Advantageously, since SO₂ pretreatment may use a relatively high amount of free SO₂ that is not associated with an added compound (e.g., alkali or carbonyl), flashing of SO₂ pretreated biomass may remove a large portion of the SO₂, and thus increase the pH of the mixture, so that the pH adjustment step(s) may not have to significantly increase the pH and/or may require less alkali.

In general, the pretreated material prepared for and fed to enzymatic hydrolysis may include the solid fraction, the liquid fraction, or some combination thereof. For example, although the primary goal of enzymatic hydrolysis is to convert the cellulose in the solid fraction to glucose, it may be advantageous to also include the liquid fraction. For example, by feeding the entire pretreated slurry (e.g., cooled and pH adjusted) to enzymatic hydrolysis the solid/liquid separation step can be avoided. Moreover, a washing step can be avoided. While washing may remove potential inhibitors and/or inactivators, and thus may increase enzyme efficiency, it may also remove fermentable sugars, and thus reduce ethanol yield. Providing little or no washing of the pretreated biomass is advantageous in that it requires less process water and provides a simpler process. Nevertheless, some washing may be advantageous in terms of providing a higher glucose yield.

In one embodiment, enzyme is added to and/or mixed with the pretreated biomass (e.g., either the solid fraction or whole) prior to feeding the pretreated biomass to the hydrolysis reactor. In one embodiment, enzyme addition is after cooling and alkali addition.

### Enzymatic Hydrolysis

In one embodiment, the pretreated material is fed to one or more enzymatic hydrolysis reactors, wherein cellulose in the solid fraction is converted to glucose. In one embodiment, the pretreated material fed to one or more enzymatic hydrolysis reactors includes washed solids (e.g., washed with water) or whole slurry (e.g., where the liquid and solid fractions are not separated). In one embodiment, the pretreated material fed to the one or more enzymatic hydrolysis reactors is pH adjusted, detoxified, and/or diluted.

In one embodiment, enzyme is added to and/or mixed with the pretreated material prior to entering the enzymatic hydrolysis reactor and/or within the enzymatic hydrolysis reactor. In one embodiment, enzyme addition is achieved by adding enzyme to a reservoir, such as a tank, to form an enzyme solution, which is then introduced to the pretreated material. In one embodiment, enzyme addition is after cooling and alkali addition. In one embodiment, enzyme addition includes the addition of cellulase.

Cellulases are enzymes that can break cellulose chains into glucose. The term “cellulase”, as used herein, includes mixtures or complexes of enzymes that act serially or synergistically to decompose cellulosic material, each of which may be produced by fungi, bacteria, or protozoans. For example, in one embodiment, the cellulase is an enzyme cocktail comprising exo-cellobiohydrolases (CBH), endoglucanases (EG), and/or β-glucosidases (βG), which can be produced by a number of plants and microorganisms. In one embodiment, the cellulase is a commercial cellulase obtained from fungi of the genera Aspergillus, Humicola, Chrysosporium, Melanocarpus, Myceliopthora, Sporotrichum or Trichoderma, or from bacteria of the genera Bacillus or Thermobifida. In addition to CBH, EG and βG, the cellulase may include several accessory enzymes that may aid in the enzymatic digestion of cellulose, including glycoside hydrolase 61 (GH61), swollenin, expansin, lucinen, and cellulose-induced protein (Cip). In one embodiment, the enzyme includes a lytic polysaccharide monooxygenase (LPMO) enzyme. For example, in one embodiment, the enzyme includes GH61. In one embodiment, the cellulase is a commercial cellulase composition that is suitable for use in the methods/processes described herein. In one embodiment, one or more cofactors are added. In one embodiment, O₂ or H₂O₂ is added. In one embodiment, ascorbic acid or some other reducing agent is added. In one embodiment, the pH is adjusted during the enzymatic hydrolysis.

In general, the enzyme dose may depend on the activity of the enzyme at the selected pH and temperature, the reaction time, the volume of the reactor, and/or other parameters. It should be appreciated that these parameters may be adjusted as desired by one of skill in the art.

In one embodiment, cellulase is added at a dosage between about 1 to 20 mg protein per gram cellulose. In one embodiment, the cellulase is added at a dosage between about 2 to 15 mg protein per gram cellulose. In one embodiment, the cellulase is added at a dosage between about 2 to 12 mg protein per gram cellulose. The protein may be quantified using either the bicinchoninic acid (BCA) assay or the Bradford assay. In one embodiment, the initial concentration of cellulose in the slurry, prior to the start of enzymatic hydrolysis, is between about 0.01% (w/w) to about 20% (w/w).

In one embodiment, the enzymatic hydrolysis is carried out at a pH and temperature that is at or near the optimum for the added enzyme. For example, in one embodiment, the enzymatic hydrolysis is carried out at one or more temperatures between about 30° C. to about 95° C. In one embodiment, the enzymatic hydrolysis is carried out at one or more temperatures between about 50° C. and about 65° C. In one embodiment, the enzymatic hydrolysis is carried out at one or more temperatures between about 45° C. and about 55° C.

In one embodiment, the enzymatic hydrolysis is carried such that the initial pH is, and/or such that the pH is maintained, between about 3.5 and about 8.0. In one embodiment, the enzymatic hydrolysis is carried out such that the initial pH is, and/or such that the pH is maintained, between about 4 and about 6. In one embodiment, the enzymatic hydrolysis is carried out such that the initial pH is, and/or such that the pH is maintained, between about 4.8 and about 5.5.

In one embodiment, the pH and temperature are adjusted to the selected range prior to the addition of the enzyme. In one embodiment, the slurry fed to hydrolysis has a solids content that is between about 10 wt. % and 25 wt. %.

In one embodiment, the enzymatic hydrolysis is carried out for a time period of about 10 to about 250 hours. In one embodiment, the enzymatic hydrolysis is carried out for a time period of about 50 to about 250 hours. In one embodiment, the enzymatic hydrolysis is carried out for at least 24 hours. In one embodiment, the enzymatic hydrolysis is carried out for at least 36 hours. In one embodiment, the enzymatic hydrolysis is carried out for at least 48 hours. In one embodiment, the enzymatic hydrolysis is carried out for at least 72 hours. In one embodiment, the enzymatic hydrolysis is carried out for at least 80 hours. In general, conducting the enzymatic hydrolysis for at least 24 hours will promote hydrolysis of both the amorphous and crystalline cellulose.

In one embodiment, the enzymatic hydrolysis includes agitation. Agitation may improve mass and/or heat transfer and may provide a more homogeneous enzyme distribution. In addition, agitation may entrain air in the slurry, which may be advantageous when the enzyme contains an lytic polysaccharide monooxygenase (LPMO). In one embodiment, air and/or oxygen is added to the hydrolysis. In one embodiment, air and/or oxygen is added to the hydrolysis using a pump or compressor in order to maintain the dissolved oxygen concentration within a range that is sufficient for the full activity of LPMOs or any other oxygen-dependent components of the catalyst used to effect hydrolysis. In one embodiment, air or oxygen is bubbled into the slurry or headspace of one or more of the hydrolysis reactors.

In one embodiment, the enzymatic hydrolysis is conducted as a batch process, a continuous process, or a combination thereof. In one embodiment, the enzymatic hydrolysis is agitated, unmixed, or a combination thereof. In one embodiment, the enzymatic hydrolysis is conducted in one or more dedicated hydrolysis reactors, connected in series or parallel. In one embodiment, the one or more hydrolysis reactors are jacketed with steam, hot water, or other heat sources.

In one embodiment, the enzymatic hydrolysis is conducted in one or more continuous stirred tank reactors (CSTRs) and/or one or more plug flow reactors (PFRs). In plug flow reactors, the slurry is pumped through a pipe or tube such that it exhibits a relatively uniform velocity profile across the diameter of the pipe/tube and such that residence time within the reactor provides the desired conversion. In one embodiment, the hydrolysis includes a plurality of hydrolysis reactors including a PFR and a CSTR in series.

In one embodiment, the enzymatic hydrolysis and fermentation are conducted in separate vessels so that each biological reaction can occur at its respective optimal temperature. In one embodiment, the enzymatic hydrolysis and fermentation are conducted in the same vessel, or series of vessels.

In one embodiment, the hydrolyzate provided by enzymatic hydrolysis is filtered to remove insoluble lignin and/or undigested cellulose.

### Fermentation

In one embodiment, the sugars produced during enzymatic hydrolysis and/or pretreatment are fermented via one or more microorganisms to produce a fermentation product (e.g., an alcohol such as ethanol or butanol). In general, the fermentation microorganism(s) may include any suitable yeast and/or bacteria.

In one embodiment, the hydrolyzate produced during enzymatic hydrolysis is subjected to a fermentation with Saccharomyces spp. yeast. For example, in one embodiment, the fermentation is carried out with Saccharomyces cerevisiae, which has the ability to utilize a wide range of hexoses such as glucose, fructose, sucrose, galactose, maltose, and maltotriose to produce a high yield of ethanol. In one embodiment, the glucose and/or other hexoses derived from the cellulose are fermented to ethanol using a wild-type Saccharomyces cerevisiae or a genetically modified yeast. In one embodiment, the fermentation is carried out with Zymomonas mobilis bacteria.

In one embodiment, the hydrolyzate produced during enzymatic hydrolysis is fermented by one or more microorganisms to produce a fermentation broth containing butanol. For example, in one embodiment the glucose produced during enzymatic hydrolysis is fermented to butanol with Clostridium acetobutylicum.

In one embodiment, one or more of the pentoses produced during the pretreatment is fermented to ethanol via one or more organisms. For example, in one embodiment, the xylose and/or arabinose produced during the pretreatment is fermented to ethanol with a yeast strain that naturally contains, or has been engineered to contain, the ability to ferment these sugars to ethanol. Examples of microbes that have been genetically modified to ferment xylose include recombinant Saccharomyces strains into which has been inserted either (a) the xylose reductase (XR) and xylitol dehydrogenase (XDH) genes from Pichia stipitis.

In one embodiment, the xylose and other pentose sugars produced during the pretreatment are fermented to xylitol by yeast strains selected from the group consisting of Candida, Pichia, Pachysolen, Hansenula, Debaryomyces, Kluyveromyces and Saccharomyces.

In general, the C6 sugar from the enzymatic hydrolysis and the C5 sugars from the liquid fraction of the pretreated biomass can be subjected to separate fermentations or a combined fermentation. For example, consider the case where the pretreated biomass is subject to a solid/liquid separation and only the solid fraction is fed to enzymatic hydrolysis. In this case, the glucose produced by enzymatic hydrolysis can be fermented on its own, or can be combined with the liquid fraction and then fermented. For example, in one embodiment, a sugar solution containing both the C5 and C6 sugars is fermented to ethanol using only Saccharomyces cerevisiae. In one embodiment, a sugar solution containing both C5 and C6 sugars is fermented to ethanol using a mixture wherein C5 utilizing and ethanol producing yeasts (e.g., such as Pichia fermentans and Pichia stipitis) are cocultured with Saccharomyces cerevisiae. In one embodiment, a sugar solution containing both C5 and C6 sugars is fermented using genetically engineered Saccharomyces yeast capable of cofermenting glucose and xylose.

In general, the dose of the microorganism(s) will depend on a number of factors, including the activity of the microorganism, the desired reaction time, the volume of the reactor, and/or other parameters. It should be appreciated that these parameters may be adjusted as desired by one of skill in the art to achieve optimal conditions. In one embodiment, the fermentation is supplemented with additional nutrients required for the growth of the fermentation microorganism. For example, yeast extract, specific amino acids, phosphate, nitrogen sources, salts, trace elements and vitamins may be added to the hydrolyzate slurry to support their growth. In one embodiment, yeast recycle is employed.

In one embodiment, the fermentation is carried out at a pH and temperature that is at or near the optimum for the added microorganism. For example, Saccharomyces cerevisiae may have optimum pH values between about 4 and about 5.5 and a temperature optimum between about 25° C. and about 35° C. In one embodiment, the fermentation is carried out at one or more temperatures between about 25° C. to about 55° C. In one embodiment, the fermentation is carried out at one or more temperatures between about 30° C. to about 35° C.

In general, the fermentation may be conducted as a batch process, a continuous process, or a fed-batch mode. For example, in one embodiment, the fermentation is conducted in continuous mode, which may offer greater productivity and lower costs. In one embodiment, the enzymatic hydrolysis may be conducted in one or more fermentation tanks, which can be connected in series or parallel. In general, the fermentation may be agitated, unmixed, or a combination thereof. For example, in one embodiment, the fermentation is conducted one or more continuous stirred tank reactors (CSTRs) and/or one or more plug flow reactors (PFRs). In one embodiment, the one or more fermentation tanks are jacketed with steam, hot water, or other heat sources.

In one embodiment, the enzymatic hydrolysis and fermentation are conducted in separate vessels so that each biological reaction can occur at its respective optimal temperature. In another embodiment, the hydrolysis (e.g., which may be also referred to as saccharification) is conducted simultaneously with the fermentation in same vessel. For example, in one embodiment, a simultaneous saccharification and fermentation (SSF) is conducted at temperature between about 35° C. and 38° C., which is a compromise between the 50° C. to 55° C. optimum for cellulase and the 25° C. to 35° C. optimum for yeast.

### Fermentation Product Recovery

In one embodiment, the fermentation product is recovered. For example, in one embodiment, the fermentation product is an alcohol and is subject to an alcohol recovery process wherein the alcohol is concentrated and/or purified from the fermented solution. In one embodiment, the fermentation broth is subject to a solid/liquid separation (e.g., filtered) and the liquid fraction is fed to a distillation system. In one embodiment, the fermentation broth, which may include unconverted cellulose, insoluble lignin, and/or other undissolved substances, is fed to the distillation system without being pre-filtered.

In one embodiment, the fermentation produces ethanol, which is recovered using one or more distillation columns that separate the ethanol from other components (e.g., water). In general, the distillation column(s) in the distillation unit may be operated in continuous or batch mode, although is typically operated in a continuous mode. Heat for the distillation process may be introduced at one or more points, either by direct steam injection or indirectly via heat exchangers. After distillation, the water remaining in the concentrated ethanol stream (i.e., vapour) may be removed from the ethanol rich vapour by molecular sieve resin, by membrane extraction, or other methods known to those of skill in the art for concentration of ethanol beyond the 95% that is typically achieved by distillation (e.g., a vapour phase drying). The vapour may then be condensed and denatured.

### Sulfur Dioxide Recovery

Excess SO₂ not consumed during the pretreatment can be recovered and/or recycled. For example, in one embodiment, SO₂ not consumed during the pretreatment is forced out of the pretreated slurry by a pressure reduction (e.g., top relief, atmospheric flash, vacuum flash, vacuum, etc.) or by a temperature increase (e.g., evaporation by heating). The SO₂ forced out of the pretreated slurry can be collected, recovered, and/or recycled within the process. In one embodiment, the SO₂ forced out of the pretreated slurry is fed to an SO₂ recovery unit. For example, in one embodiment, the slurry of pretreated material is flashed, and the flash stream, which contains the excess SO₂, is fed to a SO₂ recovery unit. In one embodiment, the SO₂ forced out of the pretreated slurry is reused directly (e.g., fed to an accumulator or the pretreatment reactor).

In general, the SO₂ recovery unit may be based on any suitable SO₂ recovery technology, as known in the art. In one embodiment, the SO₂ recovery unit includes a partial condenser, an SO₂ stripper, and/or an SO₂ scrubbing system. In one embodiment, the SO₂ recovery unit includes a SO₂ scrubbing system, which may include one or more packed absorbers (e.g., amine-based, alkali-based, or other absorbers). In one embodiment, the SO₂ recovery unit provides purified SO₂ that can be recycled for use in the pretreatment. In one embodiment, the SO₂ recovery unit provides partially purified SO₂ that can be recycled for use in the pretreatment.

In one embodiment, the recovered SO₂, which is optionally stored temporarily, is recycled directly back into the process. Advantageously, SO₂ recovery allows the recycling of sulfur within the system, and thus improves the process economics (e.g., since less SO₂ needs to be acquired for pretreatment).

Providing relatively high SO₂ loadings without a volatile solvent (e.g., ethanol) and providing limited or no added alkali may advantageously facilitate a simple flash steam recovery of sulfur dioxide. In addition, it simplifies any further purification and/or processing of the SO₂ recovered from the flash stream. Since the recovery may be relatively simple and efficient, the cost of the relatively high sulfur loading is not as limiting. Accordingly, the advantages of using a high sulfur loading for low temperature pretreatment may be exploited.

Advantageously, using a relatively high sulfur loading (e.g., greater than 20 wt. %, or greater than 25 wt. %, based on dry weight of lignocellulosic biomass) and SO₂ recovery from the flash, when at least 30% to 100% of the SO₂ in the flash is recovered and/or recycled improves the economics of the process.

Products and by-Products

In general, the process may produce a fuel from the pretreated material. In one embodiment, the process produces an alcohol (e.g., butanol or ethanol). In one embodiment, the process produces a transportation fuel. In one embodiment, the process produces ethanol for use as transportation fuel. In one embodiment, the process produces biogas or renewable natural gas (RNG) for use as a transportation fuel.

In one embodiment, the fuel includes ethanol produced by fermenting the glucose produced by enzymatically hydrolyzing the cellulose component of the lignocellulosic feedstock. However, producing one or more additional products, and/or improving the yield of ethanol using non-cellulose components (e.g., hemicellulose and/or lignin) may be advantageous.

Depending on the pretreatment conditions, in addition to unconverted cellulose, the pretreated slurry may contain hemicellulose sugars (e.g., mannose, xylose, glucose), organic acids (e.g., acetic acid), soluble lignin (e.g., lignosulfonate), soluble sugar degradation products (e.g., furfural and HMF), and/or one or more salts (e.g., sulfite salts).

In one embodiment, one or more products derived from the hemicellulose sugars are produced and/or recovered. For example, in one embodiment, wherein the pretreated slurry is subject to a solid/liquid separation and the solids are fed to enzymatic hydrolysis, the liquid fraction may be subject to separate processing. In one embodiment, the liquid fraction is pH adjusted, detoxified, and/or cooled prior to being fed to a fermenter (e.g., to produce ethanol). In this embodiment, the hemicellulose sugars may be fermented separately from the glucose produced during enzymatic hydrolysis or may be fermented with the glucose produced during enzymatic hydrolysis. Advantageously, this embodiment may improve the fermentation product (e.g., ethanol) yield.

In one embodiment, the liquid fraction is fed to an anaerobic digester, wherein the organic contents may be converted to biogas. In one embodiment, the liquid fraction is fed to a wet oxidation, wherein the organic contents may be converted to acetic acid or acetate. In one embodiment, the biogas and/or acetic acid is used as a feedstock to produce ethanol via a gas fermentation that uses carbon monoxide, carbon dioxide, and/or hydrogen as a substrate. Advantageously, this improves the ethanol yield as ethanol is produced from the cellulose component as well as from the hemicellulose and/or lignin components. In one embodiment, the biogas is used within the process in order to reduce greenhouse gas emissions. In one embodiment, the biogas is upgraded to pipeline standards and provided or allocated for transportation use or for use in producing a transportation fuel. This embodiment is particularly advantageous because in using a pretreatment liquor having a pH below about 1.5 and a relatively high SO₂ concentration, both the hemicellulose and lignin dissolution are improved, which may improve the product yield from these fractions.

In one embodiment, lignosulfonate generated during the pretreatment is recovered. In general, lignosulfonate may be recovered following pretreatment, enzymatic hydrolysis, and/or fermentation. In one embodiment, lignosulfonate is recovered for energy production (e.g., combusted). In one embodiment, lignosulfonate is recovered for producing value-added materials (e.g., a dispersing agent, a binding agent, a surfactant, an additive in oil and gas drilling, an emulsion stabilizer, an extrusion aid, to produce vanillin, for dust control applications, etc.).

In general, lignosulfonate may be recovered by any method used to produce lignosulfonate products (e.g., provided in liquid form or as a powder). For example, lignosulfonate may be recovered using a method conventionally used for recovering lignosulfonates from waste liquor (e.g., brown or red) of a sulfite pulping process. In one embodiment, lignosulfonate is recovered by precipitation and subsequent filtering, membrane separation, amine extraction, ion exchange, dialysis, or any combination thereof.

### DESCRIPTION OF SOME EMBODIMENTS

In accordance with one embodiment, a fuel is produced as follows. A batch digester having a height to diameter ratio of about 5:1 is filled with wood particles (e.g., red pine). The wood particles, which have a moisture content between 20 wt. % and 50 wt. %, are loaded with about 200 kg wood (dry basis) per m³ of digester. The digester is sealed and evacuated under vacuum. Alternatively, the digester is sealed, and the wood particles are steamed at 100° C. to 130° C. for 30 to 90 minutes to heat up the digester, drive the air out of the wood particles and/or improve penetration of SO₂. At the end of the steaming stage the condensate is drained.

Pre-prepared and pre-heated cooking liquor is added to the wood in the digester to achieve a liquid to solid ratio of about 4:1. The liquid portion includes the moisture within the wood particles, which may have a moisture content as high as about 50 wt. % after steaming. Advantageously, this quantity of cooking liquor may provide sufficient free liquor to circulate via the loop that extends outside of the digester. The cooking liquor is prepared by adding SO₂ and NaHSO₃ to provide a SO₂ concentration that is 8.4 wt. % (e.g., on liquor), where the contribution from just the SO₂ is 7.8 wt. % (on liquor). The resulting initial pH is about 1.0, measured at ambient temperature.

The slurry containing the wood particles and cooking liquor is heated via a heat exchanger coupled to the external loop, to provide a temperature of about 140° C. The heating process is continued for two hours. As the pretreatment proceeds, the pH may drop (e.g., from the formation of lignosulfonic acid). For example, without adjusting the pH, these pretreatment conditions may result in a final pH that is about 0.73 (measured at ambient temperature). The pH of the slurry being pretreated is measured via the circulation loop. For example, the pH may be measured by extracting a sample from the circulation loop, cooling it to ambient temperature, and taking the pH measurement. Alternatively, the pH may be temperature corrected.

As the pH approaches 0.9 (measured at ambient temperature), an aqueous alkali solution (e.g., NaOH) is added via the circulation loop. This may occur about 20-40 minutes into the pretreatment. In this embodiment, small alkali injections are provided every 5-15 minutes in dependence upon whether the pH is approaching 0.9. These reoccurring injections may continue for another 45 minutes or so, at which time the pH is permitted to fall or is forced to fall via the injection of an acid (e.g., HCl, H₂SO₄, H₂SO₃, oxalic acid, lignosulfonic acid).

After pretreatment, the pretreated slurry is discharged from the conical bottom section of the digester. The pressure may be reduced (e.g., flashed), the pretreated slurry cooled, pH adjusted, or otherwise prepared for enzymatic hydrolysis. The hydrolysis is conducted with a cellulase enzyme, at 50-65° C., a pH of 4.8-5.5, for 180 hours. After hydrolysis with cellulase enzyme and fermentation with yeast, the resulting ethanol is distilled and dried to provide the fuel product.

Advantageously, as a result of the pH adjustment, the glucose yield from enzymatic hydrolysis may be higher, which provides a higher ethanol yield. For example, without being bound by theory, since a relatively high amount of SO₂ is available near the start of the pretreatment, there may be rapid sulfonation of the lignin. As lignosulfonic acid is produced, and the pH begins to drop, adding alkali may prevent lignin condensation while boosting the concentration of HSO₃⁻. Advantageously, this is achieved using a single stage pretreatment (e.g., may be conducted in a single batch reactor).

Of course, the above embodiments have been provided as examples only. It will be appreciated by those of ordinary skill in the art that various modifications, alternate configurations, and/or equivalents will be employed without departing from the spirit and scope of the invention. Accordingly, the scope of the invention is therefore intended to be limited solely by the scope of the appended claims.

1. A process for producing a fuel from lignocellulosic biomass, the process comprising:
(a) obtaining a feedstock comprising lignocellulosic biomass;
(b) contacting said feedstock with sulfur dioxide and water to provide a slurry having a sulfur dioxide concentration greater than 4 wt. % on liquor and a pH less than 1.5 measured at ambient temperature;
(c) pretreating the slurry to provide a pretreated material comprising cellulose, said pretreating comprising:
(i) heating the slurry at one or more temperatures between 110° C. and 160° C., said heating conducted for a time sufficient to produce lignosulfonic acid;
(ii) adjusting the pH of the slurry containing lignosulfonic acid, said adjusting comprising removing lignosulfonic acid from the slurry, adding alkali to the slurry, or a combination thereof;
(iii) heating the pH adjusted slurry at one or more temperatures between 110° C. and 160° C.; and
(iv) optionally, repeatedly performing steps (ii) and (iii) in an alternating fashion;
(d) hydrolyzing cellulose in the pretreated material to produce glucose, said hydrolyzing comprising adding cellulase to at least the cellulose;
(e) fermenting the glucose to produce a fermentation product, said fermenting comprising adding a microorganism to at least the glucose; and
(f) recovering the fermentation product, wherein said fuel comprises the fermentation product.

2. The process according to claim 1, further comprising measuring the pH of the slurry being pretreated, wherein step (ii) is performed in dependence upon the measured pH.

3. The process according to claim 2, wherein step (ii) is performed in dependence upon the measured pH being below a predetermined limit.

4. The process according to claim 3, wherein the predetermined limit is between 0.9 and 1.1 measured at ambient temperature.

5. The process according to claim 2, further comprising monitoring a change in pH of the slurry with time as the slurry is pretreated, wherein step (ii) is performed in dependence upon a rate of change in pH exceeding a predetermined limit.

6. The process according to claim 2, wherein adjusting the pH of the slurry containing lignosulfonic acid comprises adding alkali.

7. The process according to claim 6, wherein adjusting the pH of the slurry containing lignosulfonic acid comprises adding sufficient alkali to provide the slurry being pretreated with a pH greater than 0.9 measured at ambient temperature.

8. The process according to claim 7, wherein adjusting the pH of the slurry containing lignosulfonic acid comprises adding sufficient alkali to maintain the pH of the slurry being pretreated between 0.9 and 1.1 measured at ambient temperature.

9. The process according to claim 6, wherein the alkali is added to the slurry using a circulation loop, and wherein the circulation loop is coupled to a heat exchanger for heating the slurry.

10. The process according to claim 2, wherein adjusting the pH of the slurry containing lignosulfonic acid comprises removing lignosulfonic acid.

11. The process according to claim 10, wherein removing lignosulfonic acid comprises subjecting the slurry to a filtration, pressing, washing, or any combination thereof, and wherein adjusting the pH of the slurry further comprises adding sulfur dioxide, water, or a combination thereof, in amounts sufficient to provide a sulfur dioxide concentration that is greater than 4 wt. % on liquor.

12. The process according to claim 10, wherein removing lignosulfonic acid further comprises removing cooking liquor using a circulation loop.

13. The process according to claim 12, further comprising adding water, sulfur dioxide, or a combination thereof into the pretreatment using the circulation loop in dependence upon the cooking liquor being removed.

14. The process according to claim 12, wherein step (iii) further comprises heating the slurry using a heat exchanger coupled to the circulation loop.

15. The process according to claim 1, wherein said heating in steps (i) and (iii) is conducted at one or more temperatures between 125° C. and 145° C.

16. The process according to claim 15, wherein a heating timing for the pretreatment is between 1 and 3 hours.

17. The process according to claim 16, wherein said slurry is prepared by adding alkali, and wherein an amount of alkali present in the slurry is between 0.1 wt. % (OH, on liquor) and 0.3 wt. % (OH, on liquor).

18. The process according to claim 17, wherein the feedstock is a woody feedstock.

19. The process according to claim 18, wherein the concentration of sulfur dioxide in the slurry is greater than 6 wt. % on liquor.

20. A process for producing a fuel from lignocellulosic biomass comprising:
(a) obtaining a feedstock comprising lignocellulosic biomass;
(b) providing a slurry comprising the feedstock and sulfur dioxide, and optionally including a bisulfite salt, said slurry having a concentration of sulfur dioxide that is greater than 6 wt. % on liquor and a pH less than 1.5 measured at ambient temperature;
(c) pretreating the slurry in a batch digester having a circulation loop to provide a pretreated slurry, said pretreating comprising heating the slurry between 110° C. and 160° C. for at least 30 minutes, wherein during said heating the slurry is subjected to a pH adjustment, said pH adjustment comprising adding alkali to the slurry using the circulation loop, removing lignosulfonic acid from the slurry using the circulation loop, or a combination thereof, said pH adjustment conducted in dependence upon a measured pH of the slurry being heated;
(d) obtaining a pretreated material produced from (c), said pretreated material having a solid fraction comprising cellulose and a liquid fraction comprising solubilized hemicellulose;
(e) hydrolyzing cellulose in the solid fraction to produce glucose, said hydrolyzing comprising adding cellulase to at least the solid fraction;
(f) fermenting the glucose to produce a fermentation product, said fermenting comprising adding a microorganism to at least the glucose; and
(g) recovering the fermentation product, wherein the fuel comprises the fermentation product.
